(19) United States
(12) Patent Application Publication
(10) Pub. No.: US 2022/0047599 A1
(43) Pub. Date: Feb. 17, 2022

(54) COMBINATION THERAPY OF SOLID CANCER

(71) Applicant: MOR RESEARCH APPLICATIONS LTD., Tel Aviv (IL)

(72) Inventors: Salomon STEMMER, Givat Shmuel (IL); Neta MOSKOVITS, Bet Gamliel (IL)

(21) Appl. No.: 17/417,625

(22) PCT Filed: Dec. 23, 2019
(86) PCT No.: PCT/IL2019/051400

§ 371 Date: Jun. 23, 2021

(30) Foreign Application Priority Data
Dec. 23, 2018 (IL) 263905

(51) Int. Cl. A61K 31/519; A61K 31/404; A61K 31/44; A61K 31/506; A61K 45/06; A61P 35/04

(52) U.S. Cl. CPC A61K 31/519; A61K 31/404; A61P 35/04; A61K 31/506; A61K 45/06; A61K 31/44

(57) ABSTRACT

The present invention provides methods of treating solid cancer by co-administering an inhibitor of cyclin-dependent kinase 4/6 (CDK 4/6) and multi-targeted receptor tyrosine kinase inhibitor (mt RTKI). Particular examples of CDK 4/6 inhibitor are palbociclib, abemaciclib and ribociclib and of mt RTKI are sunitinib, sorafenib and pazopanib. Administration of the combination may confer a synergic effect in treatment solid tumors. In particular synergic combinations of palbociclib with sunitinib or sorafenib are provided that synergically inhibit progression of a plurality of solid cancer types. The invention also provides pharmaceutical compositions comprising combinations of CDK 4/6 inhibitors and mt RTKIs and their use in treating solid cancer.

### FIELD OF THE INVENTION

The present invention relates to combinational therapies of solid cancer by co-administration of inhibitors of cyclin-dependent kinases 4 and 6 and inhibitors of receptor tyrosine kinases, and pharmaceutical compositions comprising said combinations of inhibitors.

### BACKGROUND OF THE INVENTION

One of the key hallmark of cancer is deregulation of the cell-cycle, resulting in aberrant cell proliferation. In normal cells a tight regulation of the cell cycle via regulatory proteins keeps the division cycles in control. Cyclin-dependent kinases 4 and 6 (CDK4/6) play a central role in this important regulation, and their inhibitors trigger cell cycle arrest. The clinical development of the CDK4/6 inhibitors has changed clinical practice in the setting of endocrine-receptor positive breast cancer. Results of pivotal phase II and III trials investigating these CDK4/6 inhibitors in patients with endocrine receptor-positive, advanced breast cancer have demonstrated a significant improvement in progression-free survival, with a safe toxicity profile. These agents were approved for use in combination with aromatase inhibitor as a first and second line of treatment of hormone positive HER2 negative metastatic breast cancer.

Most solid tumors show deregulation of multiple signaling pathways. An important mechanism in signal transduction pathways in cells is protein phosphorylation, which is carried out by protein kinases, including receptor tyrosine kinases (RTKs). These RTKs regulate the fundamental processes of proliferation, differentiation, migration, metabolism and anti-apoptotic signaling of the cell, as well as interaction with the microenvironment to regulate angiogenesis. The current trend in the development of tyrosine kinase inhibitors is the assumption that multi targeted therapy, which targets several signaling pathways simultaneously, is more effective than single targeted therapy. One example of multi-targeted receptor tyrosine kinase (RTK) inhibitor of RTK inhibitors is sunitinib, a small-molecule that was approved by the FDA for the treatment of renal cell carcinoma (RCC) and imatinib-resistant gastrointestinal stromal tumor.

The current standard of care treatments for cancer are defined and contain mostly chemotherapeutic agents, biologicals, targeted therapy and hormones (in hormone positive tumors). For patients who fail first lines (1-2 lines) of treatment, no strict guidelines are defined for further treatment in most malignancies. Combinational treatment of cancer have been recently adopted by physicians (Si-Yong Qin et al., 2018, Biomaterials, 171,178-197). Examples of such combinations are combination of chemotherapy drugs, combination of chemotherapy plus gene therapy, and chemotherapy plus immunotherapy. Bollard et al. (Gut 2017; 66:1286-1296) discussed encouraging results in preclinical models of treating hepatocellular carcinoma by palbociclib alone or in combination with sorafenib, showing an additive effect. Small et al., (Oncotarget, 2017, 8 (56), 95116-95134) showed positive results of addition of abemaciclib to sunitinib in renal cell carcinoma xenograft tumors. Uras et al., (Blood, 2016, 127 (23) pp 2890-2902) reported treatment of patients having acute myeloid leukemia harboring FLT3-IDT mutation with FLT3 inhibitors in combination with palbociclib.

There are several conditions that have to be met in order to enable the combinational therapy to succeed. Although some general considerations may be contemplated, there are actually no clear rules predicting which combination therapy will work. According to Chou (Pharmacol Rev. 58:621-681, 2006), synergism or antagonism needs to be determined, and cannot be predicted. In each case, a careful examination has to be performed. There is a long existing need for development of additional safe and proven combinational therapies to treat cancer.

### SUMMARY OF THE INVENTION

The present invention provides combinations of two separate types of anticancer drugs to achieve a synergistic therapeutic effect. Specific combinations of inhibitors of cyclin-dependent kinases 4 and 6 (CDK 4/6) together with a multi-targeted receptor tyrosine kinase inhibitor (mtRTKI) have now been found to control various types of solid tumors more efficiently than the current standard of care.

It is now disclosed that administration of inhibitors of cyclin-dependent kinases 4 and 6 (CDK 4/6) in combination with a multi-targeted receptor tyrosine kinase inhibitor (mtRTKI) provided a very efficient control of tumor growth. Administration of a combination of palbociclib and sunitinib to mice implanted with different types of malignancies not only prevented the growth of these malignancies, but in some cases reduced the initial size of the tumor. In addition, the combination treatment completely prevented the development of tumors in several different human tumors in immune deficient mice models. In fact, such combination treatment provided a synergistic anti-cancer effect. These results were completely unexpected insofar that particular combinations provided the advantageous results while other combinations were additive or less effective.

It is a well-known fact that it is difficult if not impossible to predict the effect that a combination of drugs would have. Three main effects that may be observed when two or more compounds are administered together: additive effect—the most common, a synergistic effect—the effect of a combination is more than a simple summation of individual effects, and an antagonism—the effect of a combination is less than a simple summation of individual effects.

It is shown in the present invention that combination of palbociclib and sunitinib provided a synergistic effect in a plurality of cancer models. The synergy was shown in several types of carcinoma, neuroendocrine tumor, Ewing sarcoma and in carcinosarcoma originating from different organs such as lung cancer, stomach cancer, breast cancer, ovarian cancer, colon cancer, pancreas cancer and cholangiocarcinoma. Interestingly, the synergy was not seen in hepatitis C positive hepatocellular carcinoma which showed weak to moderate additive effect. In appendix cancer model, concomitant administration of the two drugs provided effect which was worse than the effect of sunitinib alone. Combinations of other CDK 4/6 inhibitor and the mtRTKI such as a combination of ribociclib with pazopanib and combination of abemaciclib with sorafenib provided additive effects without showing synergy in some of the tested models. Combinations of palbociclib and sorafenib showed synergistic effect, e.g. in ovarian and lung cancer models. Attaining an additive effect of two anticancer drugs is an achievement by itself, which is not easy to obtain and may significantly improve the quality of treatment of patients (e.g. by reducing the dose or obtaining better effect). Obtaining a synergic effect is highly unpredictable and it is impossible to anticipate which of the combinations will provide such an effect.

According to one aspect, the present invention provides a combination of an inhibitor of cyclin-dependent kinase 4/6 (CDK 4/6) and multi-targeted receptor tyrosine kinase inhibitor (mtRTKI) for use in treating solid cancer, provided that the cancer is not hepatocellular carcinoma or renal cell carcinoma. According to some embodiments, the CDK 4/6 inhibitor is selected from palbociclib, abemaciclib and ribociclib and the mtRTKI is selected from sunitinib, sorafenib and pazopanib. According to some aspects, the present invention provides a combination of an inhibitor of cyclin-dependent kinase 4/6 (CDK 4/6) and multi-targeted receptor tyrosine kinase inhibitor (mtRTKI) for use in treating solid cancer, wherein the CDK 4/6 is palbociclib and mtRTKI is selected from sunitinib and sorafenib. According to some embodiments, the solid cancer is not hepatocellular carcinoma. According to one embodiment, the present invention provides a combination of palbociclib and sunitinib for use in treating solid cancer. According to one embodiment, the present invention provides a combination of palbociclib and sunitinib for use in treating solid cancer, provided that the cancer is not hepatocellular carcinoma. According to some embodiments, the present invention provides a combination of palbociclib and sorafenib for use in treating solid cancer, provided that the cancer is not hepatocellular carcinoma.

According to the teaching of the present invention, the combination is for treatment of cancer selected from carcinoma, neuroendocrine tumor, carcinosarcoma, Ewing sarcoma, sarcoma, lymphoma, and melanoma. According to some embodiments, the cancer is selected from lung cancer, stomach, breast cancer, ovarian cancer, neuroendocrine cancer, pancreas cancer, cholangiocarcinoma, bone cancer, bile ducts cancer, small bowel cancer, liposarcoma, brain tumor, uterus cancer, cervical cancer, head and neck, parotid, salivary gland, gallbladder cancer, bile duct cancer, adrenal cancer and colon cancer.

According to some embodiments, the present invention provides a combination of palbociclib and sunitinib for use in treating solid cancer. According to one embodiment, palbociclib is administered in a dose of from 20 to 250 mg/day and sunitinib is administered in a dose of from 10 to 125 mg/day.

According to some embodiments, the present invention provides a combination of palbociclib and sorafenib. According to one embodiment, palbociclib is administered in a dose of from 20 to 250 mg/day and sorafenib is administered in a dose of from 200 to 800 mg/day.

According to any one of the aspects and embodiments, the combination of the present invention provides a synergistic anti-cancer effect. According to some embodiments, the daily administered dose of at least one of the compounds of the combination is lower than the standard daily dose of said compound. According to one embodiment, the present invention provides a synergistic combination comprising palbociclib and sunitinib, for use in treating solid cancer, wherein the composition provides a synergic effect, provided that the cancer is not hepatocellular carcinoma or appendix cancer. According to one embodiment, the present invention provides a synergistic combination comprising palbociclib and sorafenib, for use in treating solid cancer, wherein the composition provides synergic effect, provided that the cancer is not hepatocellular carcinoma. According to one embodiment, the solid cancer is selected from carcinoma, neuroendocrine tumor, carcinosarcoma and Ewing sarcoma, lymphoma, melanoma, and sarcoma. According to yet another embodiment, wherein the cancer is selected from lung cancer, stomach cancer, breast cancer, ovarian cancer, colon cancer, neuroendocrine cancer, pancreas cancer, and cholangiocarcinoma and optionally, bone cancer, liposarcoma, and adrenal cancer.

According to another aspect, the present invention provides a pharmaceutical composition comprising an inhibitor of cyclin-dependent kinase 4/6 (CDK 4/6) and a multi-targeted receptor tyrosine kinase inhibitor (mtRTKI), wherein the CDK 4/6 inhibitor is selected from palbociclib, abemaciclib and ribociclib and the mtRTKI is selected from sunitinib, sorafenib and pazopanib. According to some embodiments, the present invention provides a pharmaceutical composition comprising palbociclib and sunitinib, e.g. from 20 to 150 mg/day palbociclib and from 5 to 75 mg/day of sunitinib. According to other embodiments, the present invention provides a pharmaceutical composition comprising palbociclib and sorafenib, e.g. from 20 to 150 mg/day palbociclib and from 50 to 800 mg sorafenib.

According to other embodiments, the present invention provides a pharmaceutical composition comprising ribociclib and pazopanib, e.g. from 50 to 300 mg ribociclib and from 100 to 500 mg of pazopanib.

According to further embodiments, the present invention provides a pharmaceutical composition comprising abemaciclib and sorafenib, e.g. from 20 to 300 mg abemaciclib and from 50 to 300 mg of sorafenib.

According to some embodiments, the pharmaceutical compositions of the present invention are for use in treating solid cancer, e.g. for treatment of lung cancer, stomach, small bowel cancer, breast cancer, ovarian cancer, neuroendocrine cancer, pancreas cancer, cholangiocarcinoma, bone cancer, gallbladder cancer, bile duct cancer, liposarcoma, brain tumor, uterus cancer, cervical cancer, head and neck, parotid, salivary gland, adrenal cancer and colon cancer. According to some embodiments, the cancer is selected from carcinoma, neuroendocrine tumor, lymphoma, carcinosarcoma, Ewing sarcoma, melanoma and sarcoma.

According to some embodiments, the pharmaceutical composition provides a synergic anticancer effect. According to some embodiments, it is provided that the cancer is not a hepatocellular carcinoma. According to other embodiments, it is provided that the cancer is not renal cell carcinoma.

Thus, according to some embodiments, the present invention provides a synergic pharmaceutical composition comprising palbociclib and multi-targeted receptor tyrosine kinase inhibitor selected from sunitinib and sorafenib. According to some embodiments, the pharmaceutical composition comprising palbociclib and a multi-targeted receptor tyrosine kinase inhibitor selected from sunitinib and sorafenib is for use in treating solid cancer, provided that the cancer is not hepatocellular carcinoma. According to some embodiments, the cancer is selected from lung cancer, stomach cancer, breast cancer, ovarian cancer, colon cancer, neuroendocrine cancer, pancreas cancer, cholangiocarcinoma, bone cancer, liposarcoma, and adrenal cancer. According to other embodiments, the cancer is selected from carcinoma, neuroendocrine tumor, carcinosarcoma, Ewing sarcoma, lymphoma, sarcoma and melanoma. According to some embodiments, the cancer is selected from carcinoma, neuroendocrine tumor, carcinosarcoma, Ewing sarcoma, sarcoma and melanoma. According to some embodiments, the cancer is selected from carcinoma, neuroendocrine tumor, carcinosarcoma, Ewing sarcoma, and sarcoma. According to some embodiments, the cancer is selected from carcinoma, neuroendocrine tumor, carcinosarcoma, and Ewing sarcoma.

According to another aspect, the present invention provides a method of treating a solid cancer in a subject in need thereof comprising co-administering an inhibitor of cyclin-dependent kinase 4/6 (CDK 4/6) selected from palbociclib, abemaciclib and ribociclib and multi-targeted receptor tyrosine kinase inhibitor (mtRTKI) selected from sunitinib, sorafenib and pazopanib. According to some embodiments, it is provided that the cancer is not hepatocellular carcinoma and/or not a renal cell carcinoma. According to one embodiment, the method provides administering palbociclib and sunitinib, wherein the method provides a synergic anticancer effect. According to another embodiment, the method provides administering palbociclib and sorafenib, wherein the method provides a synergic anticancer effect. According to one embodiment, the present invention provides a method of treating a solid cancer in a subject in need thereof comprising co-administering an inhibitor of cyclin-dependent kinase 4/6 (CDK 4/6) palbociclib, and multi-targeted receptor tyrosine kinase inhibitor (mtRTKI) sunitinib. According to another embodiment, the present invention provides a method of treating a solid cancer in a subject in need thereof comprising co-administering an inhibitor of cyclin-dependent kinase 4/6 (CDK 4/6) palbociclib, and multi-targeted receptor tyrosine kinase inhibitor (mtRTKI) sorafenib, provided that the cancer is not hepatocellular carcinoma.

According to a further aspect, the present invention provides a kit comprising at least one inhibitor of cyclin-dependent kinase 4/6 (CDK 4/6) and at least one multi-targeted receptor tyrosine kinase inhibitor (mtRTKI), and instructions for use, wherein the CDK 4/6 is palbociclib and mtRTKI is selected from sunitinib and sorafenib. According to one embodiments, the CDK 4/6 inhibitor and the mtRTKI are present as a pharmaceutical composition.

### BRIEF DESCRIPTION OF DRAWINGS

FIG. 1 shows the effect of a combo treatment with palbociclib and sunitinib in colon cancer model (adenocarcinoma).

FIG. 2 shows influence of doses of palbociclib and sunitinib in combination on tumor size in colon cancer model

FIG. 3 shows effect of a combo treatment with palbociclib and sunitinib in colon cancer model.

FIG. 4 shows influence of doses of palbociclib and sunitinib in combination on tumor size in colon cancer model.

FIG. 5 shows effect of a combo treatment with palbociclib and sunitinib in colon cancer model.

FIG. 6A shows the effect of a combo treatment with palbociclib and sunitinib in stomach cancer model. FIG. 6B shows the effect of the standard treatment of oxaliplatin+5FU on the same cancer tissue.

FIG. 7 shows the effect of a combo treatment with: ribociclib and pazopanib or abemaciclib and sorafenib in stomach cancer model.

FIG. 8 shows the effect of combo treatments in breast cancer.

FIG. 9 shows the effect of a combo treatment with palbociclib and sunitinib in 5FU resistant breast cancer model.

FIG. 10 shows the effect of combo treatments in breast cancer.

FIG. 11 shows the effect of combo treatment of palbociclib and sunitinib in breast cancer.

FIG. 12 shows the effect of a combo treatment of palbociclib and sunitinib in breast cancer.

FIG. 13 shows influence of doses of palbociclib and sunitinib in combination on tumor size in breast cancer model.

FIG. 14 shows the effect of a combo treatment of palbociclib and sunitinib in lung cancer.

FIG. 15 shows the effect of a combo treatment of palbociclib and sunitinib in lung cancer. FIG. 15A shows progression of tumor with time of different groups. FIG. 15B shows tumors excised at the last day of the experiment (day 60) from mice of different treatment group (M represent one mouse).

FIG. 16 shows the effect of a combo treatment of palbociclib and sunitinib in ovarian cancer model.

FIG. 17 shows the effect of combo treatments in ovarian cancer model.

FIG. 18 shows the effect of a combo treatment of palbociclib and sunitinib in carcinosarcoma model.

FIG. 19 shows the effect of a combo treatment of palbociclib and sunitinib in pancreatic cancer.

FIG. 20 shows influence of doses of palbociclib and sunitinib in combination on tumor size in pancreatic cancer model.

FIG. 21 shows the effect of a combo treatment of palbociclib and sunitinib in cholangiocarcinoma.

FIG. 22 shows influence of doses of palbociclib and sunitinib in combination on tumor size in cholangiocarcinoma cancer model.

FIG. 23 shows the effect of a combo treatment of palbociclib and sunitinib in hepatocellular carcinoma.

FIG. 24 shows the effect of a combo treatment of palbociclib and sunitinib in appendix carcinoma.

FIG. 25 shows the effect of a combo treatment of ribociclib and pazopanib in appendix carcinoma.

FIG. 26 shows the effect of a combo treatment of palbociclib and sunitinib in Ewing sarcoma.

### DETAILED DESCRIPTION OF THE INVENTION

The present invention provides efficient combo treatments of a wide range of cancer malignancies based on a combination of an inhibitor of cyclin-dependent kinase 4/6 and an inhibitor of receptor tyrosine kinase.

According to one aspect, the present invention provides a combination of an inhibitor of cyclin-dependent kinase 4/6 (CDK 4/6) and multi-targeted receptor tyrosine kinase inhibitor (mtRTKI) for use in treating solid cancer. According to some embodiments, it is provided that the cancer is not hepatocellular carcinoma. According to other embodiments, it is provided that the cancer is not a renal cell carcinoma. According to one embodiment, the CDK 4/6 inhibitor is selected from palbociclib, abemaciclib and ribociclib. According to another embodiment, the mtTKI is selected from sunitinib, sorafenib and pazopanib. According to yet another embodiment, the CDK 4/6 inhibitor is selected from palbociclib, abemaciclib and ribociclib and the mtTKI is selected from sunitinib, sorafenib, pazopanib. Thus, the present invention provides a provides a combination of an inhibitor of CDK 4/6 selected from palbociclib, abemaciclib and ribociclib and an mtRTKI selected from sunitinib, sorafenib and pazopanib, for use in treating solid cancer, provided that the cancer is not hepatocellular carcinoma and not a renal cell carcinoma.

The terms “cyclin-dependent kinase 4”, “cell division protein kinase 4” and “CDK4” are used herein interchangeably and refer to a human enzyme encoded by the CDK4 gene. The terms “cyclin-dependent kinase 4”, “cell division protein kinase 4” and CDK4 are used herein interchangeably and refer to a human enzyme having EC 2.7.11.22 and encoded by CDK4 gene. The terms “cyclin-dependent kinase 6”, and “CDK6” are used herein interchangeably and refer to human enzyme having EC 2.7.11.22 and encoded by CDK6 gene. The terms “inhibitor of cyclin-dependent kinase 4/6”, “inhibitor of CDK 4/6” and “CDK 4/6 inhibitor” are used herein interchangeably and refer to an inhibitor that inhibit the activity of both CDK4 and CDK6 enzymes.

The terms “receptor tyrosine kinase” and “RTK” are used herein interchangeably and refer to human receptor tyrosine kinases having EC number 2.7.10.1. The term “multi-targeted receptor tyrosine kinase inhibitor” and “mtRTKI” are used herein interchangeably and refer to inhibitors that inhibit two or more receptor tyrosine kinases.

According to one embodiment, the CDK 4/6 inhibitor is selected from palbociclib, abemaciclib and ribociclib and the mtRTKI is selected from sunitinib, sorafenib and pazopanib, provided that the solid cancer is not hepatocellular carcinoma or renal cell carcinoma.

According to one embodiment, the combination is a combination of palbociclib and sunitinib. According to one embodiment, the combination is a combination of palbociclib and sorafenib. According to another embodiment, the combination is a combination of palbociclib and pazopanib. According to a further embodiment, the combination is a combination of abemaciclib and sorafenib. According to a certain embodiment, the combination is a combination of abemaciclib and pazopanib. According to yet another embodiment, the combination is a combination of abemaciclib and sunitinib. According to some embodiments, the combination is a combination of ribociclib and sunitinib. According to other embodiments, the combination is a combination of ribociclib and sorafenib. According to one embodiment, the combination is a combination of ribociclib and pazopanib.

The terms “treating” or “treatment of” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s). The term “treating” with respect to cancer should be understood to e.g. encompass treatment resulting in a decrease in tumor size; a decrease in rate of tumor growth; stasis of tumor size; a decrease in the number of metastasis; a decrease in the number of additional metastasis; a decrease in invasiveness of the cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as decrease in the angiogenesis induced by the cancer, inhibition of growth and proliferation of cancer cells and so forth. The term “treating cancer” as used herein should also be understood to encompass prophylaxis such as prevention as cancer reoccurs after previous treatment (including surgical removal) and prevention of cancer in an individual prone (genetically, due to life style, chronic inflammation and so forth) to develop cancer. As used herein, “prevention of cancer” is thus to be understood to include prevention of metastases, for example after surgical procedures or after chemotherapy.

As used herein, the term “cancer” refers to all types of cancer, neoplasm or malignant tumors found in mammals. As used herein, the term “solid cancer” refers to a mass-type cancer formed in an organ, unlike blood cancer. The term solid cancer includes lymphomas, melanomas, neuroendocrine tumors, carcinomas, carcinosarcoma and Ewing sarcoma and sarcomas. “Sarcomas” are cancers of the connective tissue, cartilage, bone, muscle, and so on. “Carcinomas” are cancers of epithelial (lining) cells. “Adenocarcinoma” refers to carcinoma derived from cells of glandular origin. “Neuroendocrine” tumors is derived from cells that release hormones into the blood. The terms “cancer” and “tumor” are used interchangeably throughout the subject specification. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, thyroid cancer, prostate cancer, breast cancer (e.g. triple negative, ER positive, ER negative, chemotherapy resistant, herceptin resistant, HER2 positive, doxorubicin resistant, tamoxifen resistant, ductal carcinoma including palbociclib resistant ductal carcinoma, lobular carcinoma, primary, metastatic), ovarian cancer, pancreatic cancer, lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, lymphoma, or B cell lymphoma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or medulloblastoma, Hodgkin's disease, non-Hodgkin's lymphoma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, Paget's disease of the nipple, phyllodes tumors, lobular carcinoma, ductal carcinoma, cancer of the pancreatic stellate cells, cancer of the hepatic stellate cells, or prostate cancer.

According to one embodiment, the combination of the present invention is for use in treating solid cancer, wherein the solid cancer is selected from carcinoma, neuroendocrine tumor, Ewing sarcoma, carcinosarcoma, lymphoma, melanoma, and sarcoma. According to one embodiment, the solid cancer is selected from lung cancer, stomach, small bowel cancer, breast cancer, ovarian cancer, neuroendocrine cancer, pancreas cancer, cholangiocarcinoma, gallbladder cancer, bile duct cancer, colon cancer, bone cancer, liposarcoma, brain tumor, uterus cancer, cervical cancer, head and neck, parotid, salivary gland, and adrenal cancer. According to one embodiment, the solid cancer is originated from lung cancer, stomach, small bowel, breast, ovary, endocrine system, pancreas, gallbladder cancer, bile duct cancer, colon cancer, bone cancer, fat cells, brain, uterus, cervix, head and neck, parotid, salivary, or adrenal gland.

According to one embodiment, the present invention provides a combination of CDK 4/6 inhibitor and mtRTKI for use in treating carcinoma. According to one embodiment, the carcinoma is selected from medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum. According to some embodiments, the combination is palbociclib and sunitinib. According to another embodiment, the combination is palbociclib and sorafenib.

According to some embodiments, the CDK 4/6 inhibitor is selected from palbociclib, abemaciclib and ribociclib and the mtRTKI is selected from sunitinib, sorafenib and pazopanib.

According to some embodiments, the present invention provides a combination of an inhibitor of cyclin-dependent kinase 4/6 (CDK 4/6) and multi-targeted receptor tyrosine kinase inhibitor (mtRTKI) for use in treating solid cancer, wherein the CDK 4/6 is palbociclib and mtRTKI is selected from sunitinib and sorafenib. According to some embodiments the cancer is not hepatocellular carcinoma. According to some embodiments, the cancer is selected from lung cancer, stomach cancer, breast cancer, ovarian cancer, colon cancer, neuroendocrine cancer, pancreas cancer, cholangiocarcinoma, bone cancer, liposarcoma, gallbladder cancer, bile duct cancer and adrenal cancer. According to other embodiments, the solid cancer is selected from carcinoma, neuroendocrine tumor, Ewing sarcoma, lymphoma, melanoma, and sarcoma. According to some embodiments, the solid cancer is selected from carcinoma, neuroendocrine tumor, Ewing sarcoma, melanoma, and sarcoma. According to some embodiments, the solid cancer is selected from carcinoma, neuroendocrine tumor, Ewing sarcoma, and sarcoma.

According to some embodiments, palbociclib is administered in the dose of 25 to 250, 30 to 225, 35 to 200, 40 to 175, 50 to 150, 55 to 130, 60 to 125, or 75 to 100 mg/day.

According to other embodiments, the sunitinib is administered in the dose of 10 to 125, 15 to 110, 20 to 100, 25 to 75, 30 to 70, 35 to 60 or 40 to 55 mg/day.

According to some embodiments, ribociclib is administered in the dose of from 50 to 800, 60 to 700, 70 to 600, 80 to 500, 100 to 400 or 200 to 300 mg/day.

According to one embodiment, pazopanib is administered in the dose of 100 to 950, 150 to 900, 200 to 850, 250 or 300 to 800, 400 to 700, or 500 to 600 mg/day.

According to some embodiments, abemaciclib is administered in the dose of from 100 to 800, 150 to 700, 200 to 600, or 300 to 500 mg/day.

According to one embodiment, sorafenib is administered in the dose of 100 to 950, 150 to 900, 200 to 850, 250 or 300 to 800, 400 to 700, or 500 to 600 mg/day.

According to one embodiment, the combination is for use in treating solid cancer selected from lymphoma, melanoma, neuroendocrine tumor, carcinoma, Ewing sarcoma, carcinosarcoma and sarcoma. According to one embodiment, the combination is for use in treating carcinoma. According to one embodiment, carcinoma is an adenocarcinoma. According to another embodiment, the combination is for use in treating neuroendocrine tumor. According to yet another embodiment, the combination is for use in treating sarcoma. According to a further embodiment, the combination is for use in treating carcinosarcoma. According to some embodiments, the combination is for use in treating Ewing sarcoma. According to one embodiment, the solid cancer is selected from lung cancer, stomach, colon cancer, breast cancer, ovarian cancer, neuroendocrine cancer, pancreas cancer, cholangiocarcinoma and bone cancer. According to one embodiment, the solid cancer is lung cancer. According to one embodiment, the solid cancer is stomach cancer. According to one embodiment, the solid cancer is stomach cancer. According to one embodiment, the solid cancer is colon cancer. According to one embodiment, the solid cancer is breast cancer. According to one embodiment, the solid cancer is ovarian cancer. According to one embodiment, the solid cancer is neuroendocrine cancer. According to one embodiment, the solid cancer is pancreas cancer. According to one embodiment, the solid cancer is cholangiocarcinoma cancer. According to another embodiment, the solid cancer is bone cancer. According to another embodiment, the solid cancer is gallbladder or bile duct cancer.

According to some embodiments, the present invention provides a combination of palbociclib and sunitinib for use in treating a solid cancer. According to one embodiment, palbociclib is administered in a dose of from 20 to 300 mg/day. According to some embodiments, palbociclib is administered in the dose of 25 to 250, 30 to 225, 35 to 200, 40 to 175, 50 to 150, 55 to 130, 60 to 125, or 75 to 100 mg/day. According to another embodiment, palbociclib is administered in the dose of 25 to 150, 50 to 125 or 75 to 100 mg/day. According to another embodiment, sunitinib is administered in the dose of from 5 to 150 mg/day. According to one embodiment, sunitinib is administered in the dose of 10 to 125, 15 to 110, 20 to 100, 25 to 75, 30 to 70, 35 to 60 or 40 to 55 mg/day. According to another embodiment, sunitinib is administered in the dose of 5 to 50 mg/day, 10 to 40 mg/day or 20 to 30 mg/day. According to one embodiment, palbociclib is administered in the dose of 25 to 250 mg/day and sunitinib is administered in the dose of 10 to 125. According to one embodiment, palbociclib is administered in the dose of 25 to 150 mg/day and sunitinib is administered in the dose of 5 to 50 mg/day. According to one embodiment, the combination is for use in treating solid cancer selected from lymphoma, melanoma, neuroendocrine tumor, carcinoma, Ewing sarcoma, carcinosarcoma and sarcoma. According to one embodiment, the combination is for use in treating carcinoma such as adenocarcinoma. According to another embodiment, the combination is for use in treating neuroendocrine tumor. According to yet another embodiment, the combination is for use in treating sarcoma. According to a further embodiment, the combination is for use in treating carcinosarcoma. According to some embodiments, the combination is for use in treating Ewing sarcoma. According to one embodiment, the solid cancer is selected from lung cancer, stomach, colon cancer, breast cancer, ovarian cancer, neuroendocrine cancer, pancreas cancer, cholangiocarcinoma and bone cancer. According to one embodiment, the solid cancer is lung cancer. According to one embodiment, the solid cancer is stomach cancer. According to one embodiment, the solid cancer is stomach cancer. According to one embodiment, the solid cancer is colon cancer. According to one embodiment, the solid cancer is breast cancer. According to one embodiment, the solid cancer is ovarian cancer. According to one embodiment, the solid cancer is neuroendocrine cancer. According to one embodiment, the solid cancer is pancreas cancer. According to one embodiment, the solid cancer is cholangiocarcinoma cancer. According to another embodiment, the solid cancer is bone cancer. According to another embodiment, the solid cancer is gallbladder or bile duct cancer. Other examples are liposarcoma, brain tumor, uterus cancer, cervical cancer, head and neck, parotid, salivary gland, adrenal cancer and small bowel cancer. According to one embodiment, the breast cancer is 5FU resistant or palbociclib resistant breast cancer. According to some embodiments, the cancer is not hepatocellular carcinoma. According to another embodiment, the cancer is not appendix cancer.

According to some embodiments, the present invention provides a combination of palbociclib and sorafenib for use in treating solid cancer. According to one embodiment, palbociclib is administered in a dose of from 20 to 300 mg/day. According to some embodiments, palbociclib is administered in the dose of 25 to 250, 30 to 225, 35 to 200, 40 to 175, 50 to 150, 55 to 130, 60 to 125, or 75 to 100 mg/day. According to another embodiment, palbociclib is administered in the dose of 25 to 150, 50 to 125 or 75 to 100 mg/day. According to one embodiment, sorafenib is administered in the dose of 100 to 950, 150 to 900, 200 to 850, 250 or 300 to 800, 400 to 700, or 500 to 600 mg/day. According to another embodiment, sorafenib is administered in the dose of 100 to 900, 150 to 850, 200 to 800, or 300 to 700 mg/day. According to one embodiment, palbociclib is administered in the dose of 25 to 150 mg/day and sorafenib is administered in the dose of from 50 to 1200 mg/day. According to one embodiment, the combination is for use in treating solid cancer selected from lymphoma, melanoma, neuroendocrine tumor, carcinoma and sarcoma. According to one embodiment, the combination is for use in treating carcinoma such as adenocarcinoma. According to another embodiment, the combination is for use in treating neuroendocrine tumor. According to yet another embodiment, the combination is for use in treating sarcoma and/or carcinosarcoma. According to a further embodiment, the combination is for use in treating Ewing sarcoma. According to one embodiment, the solid cancer is selected from lung cancer, stomach, colon cancer, breast cancer, ovarian cancer, neuroendocrine cancer, pancreas cancer, bone and cholangiocarcinoma. According to one embodiment, the solid cancer is lung cancer. According to one embodiment, the solid cancer is stomach cancer. According to one embodiment, the solid cancer is stomach cancer. According to one embodiment, the solid cancer is colon cancer. According to one embodiment, the solid cancer is breast cancer. According to one embodiment, the solid cancer is ovarian cancer. According to one embodiment, the solid cancer is neuroendocrine cancer. According to one embodiment, the solid cancer is pancreas cancer. According to one embodiment, the solid cancer is cholangiocarcinoma cancer. According to one embodiment, the solid cancer is bone cancer. Other examples are liposarcoma, brain tumor, uterus cancer, cervical cancer, head and neck, parotid, salivary gland, adrenal cancer and small bowel cancer. According to one embodiment, the breast cancer is 5FU resistant or palbociclib resistant breast cancer. According to some embodiments, the cancer is not hepatocellular carcinoma.

According to some embodiments, the present invention provides a combination of ribociclib and pazopanib for use in treating solid cancer. According to one embodiment, ribociclib is administered in a dose of from 50 to 1000 mg/day. According to some embodiments, ribociclib is administered in the dose of from 50 to 800, 60 to 700, 70 to 600, 80 to 500, 100 to 400 or 200 to 300 mg/day. According to another embodiment, ribociclib is administered in the dose of 50 to 600, 100 to 500 mg/day 150 to 450 mg/day or 200 to 400 mg/day. According to one embodiment, the dose is an initial dose for treatment. According to another embodiment, pazopanib is administered in the dose of from 50 to 1000 mg/day. According to one embodiment, pazopanib is administered in the dose of 100 to 950, 150 to 900, 200 to 850, 250 or 300 to 800, 400 to 700, or 500 to 600 mg/day. According to one embodiment, ribociclib is administered in a dose of from 50 to 1000 mg/day and pazopanib is administered in the dose of from 50 to 1000 mg/day. According to one embodiment, ribociclib is administered in the dose of 50 to 600 and pazopanib is administered in the dose of 200 to 800 mg/day. According to one embodiment, the combination is for use in treating solid cancer selected from lymphoma, melanoma, neuroendocrine tumor, carcinoma and sarcoma. According to one embodiment, the combination is for use in treating carcinoma such as adenocarcinoma. According to another embodiment, the combination is for use in treating neuroendocrine tumor. According to yet another embodiment, the combination is for use in treating sarcoma and/or carcinosarcoma. According to some embodiments, the combination is for use in treating Ewing sarcoma. According to one embodiment, the solid cancer is selected from lung cancer, stomach, colon cancer, breast cancer, ovarian cancer, neuroendocrine cancer, pancreas cancer, cholangiocarcinoma, gallbladder cancer, bile duct cancer and bone cancer. According to one embodiment, the solid cancer is lung cancer. According to one embodiment, the solid cancer is stomach cancer. According to one embodiment, the solid cancer is stomach cancer. According to one embodiment, the solid cancer is colon cancer. According to one embodiment, the solid cancer is breast cancer. According to one embodiment, the solid cancer is ovarian cancer. According to one embodiment, the solid cancer is neuroendocrine cancer. According to one embodiment, the solid cancer is pancreas cancer. According to one embodiment, the solid cancer is cholangiocarcinoma cancer. According to another embodiment, the solid cancer is bone cancer. Other examples are liposarcoma, brain tumor, uterus cancer, cervical cancer, head and neck, parotid, salivary gland, adrenal cancer and small bowel cancer. According to one embodiment, the breast cancer is 5FU resistant or palbociclib resistant breast cancer.

According to some embodiments, the present invention provides a combination of abemaciclib and sorafenib for treating solid cancer. According to one embodiment, abemaciclib is administered in the dose of from 50 to 1000 mg/day. According to some embodiments, abemaciclib is administered in the dose of from 100 to 800, 150 to 700, 200 to 600, or 300 to 500 mg/day. According to another embodiment, abemaciclib is administered in the dose of 100 to 500, 150 to 450 mg/day 200 to 400 mg/day or 250 to 350 mg/day. According to one embodiment, the dose is an initial dose for treatment. According to another embodiment, sorafenib is administered in the dose of from 50 to 1200 mg/day. According to one embodiment, sorafenib is administered in the dose of 100 to 950, 150 to 900, 200 to 850, 250 or 300 to 800, 400 to 700, or 500 to 600 mg/day. According to another embodiment, sorafenib is administered in the dose of 100 to 900, 150 to 850, 200 to 800, or 300 to 700 mg/day. According to one embodiment, abemaciclib is administered in the dose of from 50 to 1000 mg/day and sorafenib is administered in the dose of from 50 to 1200 mg/day. According to one embodiment, the combination is for use in treating solid cancer selected from lymphoma, melanoma, neuroendocrine tumor, carcinoma and sarcoma. According to one embodiment, the combination is for use in treating carcinoma such as adenocarcinoma. According to another embodiment, the combination is for use in treating neuroendocrine tumor. According to yet another embodiment, the combination is for use in treating sarcoma and/or carcinosarcoma. According to one embodiment, the solid cancer is selected from lung cancer, stomach, colon cancer, breast cancer, ovarian cancer, neuroendocrine cancer, pancreas cancer, and cholangiocarcinoma. According to one embodiment, the solid cancer is lung cancer. According to one embodiment, the solid cancer is stomach cancer. According to one embodiment, the solid cancer is stomach cancer. According to one embodiment, the solid cancer is colon cancer. According to one embodiment, the solid cancer is breast cancer. According to one embodiment, the solid cancer is ovarian cancer. According to one embodiment, the solid cancer is neuroendocrine cancer. According to one embodiment, the solid cancer is pancreas cancer. According to one embodiment, the solid cancer is cholangiocarcinoma cancer. According to some embodiments, the combination is for use in treating Ewing sarcoma. Other examples are liposarcoma, brain tumor, uterus cancer, cervical cancer, head and neck, parotid, salivary gland, adrenal cancer and small bowel cancer. According to one embodiment, the breast cancer is 5FU resistant or palbociclib resistant breast cancer. According to some embodiments, the cancer is not renal cell carcinoma.

According to some embodiments, the combination provides an additive anticancer effect.

According to any one of the above embodiments, the combination provides synergistic anti-cancer effect. According to one embodiment, the combination of CDK 4/6 inhibitor and the mtRTKI provides a synergistic anti-cancer effect. Thus, the combination of the present intention is a synergic anticancer combination. According to one embodiment, the combination of palbociclib and sunitinib, provides a synergistic anti-cancer effect. According to one embodiment, the cancer is not hepatocellular carcinoma. According to another embodiment, the cancer is not appendix cancer. According to another embodiment, the combination of palbociclib and sorafenib provides a synergistic anti-cancer effect, wherein the cancer is not hepatocellular carcinoma.

As used herein, the term “synergistic anticancer effect” means that the combination of the components of the combination exhibits greater anticancer effect or activity than the additive effect or activity provided when each component of the combination is applied alone. The term “synergic” and “synergistic” are used herein interchangeably. A synergistic effect of a combination of therapies permits the use of lower dosages of one or more of the therapeutic agent(s) and/or less frequent administration of the agent(s) to a subject with a disease or disorder, e.g., a proliferative disorder. The ability to utilize lower the dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

According to one embodiment, the present invention provides combination of palbociclib and sunitinib for use in treating solid cancer, wherein the combination provides a synergic anticancer effect, provided that the cancer is not hepatocellular carcinoma or appendix cancer. According to some embodiments, the present invention provides a synergic combination comprising palbociclib and sunitinib for use in treating a solid cancer selected from carcinoma, neuroendocrine tumor, Ewing sarcoma, carcinosarcoma, lymphoma, melanoma, and sarcoma.

According to some embodiments, the solid cancer is carcinoma, as define herein above. According to another embodiment, the solid cancer is adenocarcinoma. According to a further embodiment, the solid cancer is Ewing sarcoma. According to a further embodiment, the cancer is selected from lung cancer, stomach cancer, breast cancer, ovarian cancer, colon cancer, neuroendocrine cancer, bone cancer, gallbladder cancer, bile duct cancer, pancreas cancer, and cholangiocarcinoma. According to some embodiments, the cancer is characterized by KRAS mutation. According to one embodiment, KRAS mutation is G12V mutation. According to other embodiments, the cancer is characterized by HER2 negative mutation. According to some embodiments, the cancer is estrogen receptor positive (ER+). According to yet another embodiment, the cancer is characterized by presence BRCA1 gene. According to yet another embodiment, the cancer expresses wild-type RAS protein.

According to some embodiments, the present invention provides a synergic combination comprising palbociclib and sunitinib for use in treating neuroendocrine cancer, wherein the combination provides a synergistic effect. According to some embodiments, the present invention provides a synergic combination comprising palbociclib and sunitinib for use in treating sarcoma, wherein the combination provides a synergistic effect. According to some embodiments, the present invention provides a synergic combination comprising palbociclib and sunitinib for use in treating carcinosarcoma, wherein the combination provides a synergistic effect. According to some embodiments, the present invention provides a synergic combination comprising palbociclib and sunitinib for use in treating Ewing sarcoma, wherein the combination provides a synergistic effect.

According to some embodiments, the present invention provides a synergic combination comprising palbociclib and sunitinib for use in treating stomach cancer, wherein the combination provides a synergistic effect. According to one embodiment, the stomach cancer is HER2 negative adenocarcinoma.

According to some embodiments, the present invention provides a synergic combination comprising palbociclib and sunitinib for use in treating breast cancer, wherein the combination provides a synergistic effect. According to one embodiment, the breast cancer is estrogen receptor-positive (ER+) carcinoma. According to another embodiment, the breast cancer is palbociclib resistant carcinoma. According to yet another embodiment, the breast cancer is ER+ and palbociclib resistant carcinoma. According to some embodiments, the breast cancer is FU resistant carcinoma. According to some embodiments, the breast cancer is HER2 negative. According to yet another embodiment, the breast cancer is triple negative breast cancer (TNBC), i.e. estrogen receptor negative, progesterone receptor negative and HER2 negative. According to some embodiments, the cancer is metastatic cancer.

According to another embodiment, the present invention provides a synergic combination comprising palbociclib and sunitinib for use in treating colon cancer, wherein the combination provides a synergistic effect. According to some embodiments, the colon cancer is characterized by KRAS mutation. According to one embodiment, the KRAS mutation is G12V mutation. According to yet another embodiment, the colon cancer is an adenocarcinoma with wild type RAS. According to yet another embodiment, the cancer is a metastatic adenocarcinoma.

According to another embodiment, the present invention provides a synergic combination comprising palbociclib and sunitinib, for use in treating lung cancer, wherein the combination provides a synergistic effect. According to one embodiment, the lung cancer is carcinoma. According to another embodiment, the lung cancer is adenocarcinoma. According to a further embodiment, the lung cancer is carcinoma. According to some embodiments, the lung cancer is an adenocarcinoma characterized by KRAS mutation. According to one embodiment, the KRAS mutation is G12V mutation.

According to another embodiment, the present invention provides a synergic combination comprising palbociclib and sunitinib for use in treating ovarian cancer, wherein the combination provides a synergistic effect. According to one embodiment, the ovarian cancer is carcinoma. According to one embodiment, the carcinoma is characterized by presence or BRCA1 gene. According to another embodiment, the ovarian cancer is neuroendocrine ovarian cancer. According to yet another embodiment, the neuroendocrine or carcinoma is a metastatic one.

According to another embodiment, the present invention provides a synergic combination comprising palbociclib and sunitinib for use in treating cholangiocarcinoma, wherein the combination provides a synergistic effect.

According to another embodiment, the present invention provides a synergic combination comprising palbociclib and sunitinib for use in treating pancreatic cancer, wherein the combination provides a synergistic effect. According to one embodiment, the pancreatic cancer is adenocarcinoma. According to one embodiment, the adenocarcinoma is metastatic adenocarcinoma.

According to another embodiment, the present invention provides a synergic combination comprising palbociclib and sunitinib for use in treating carcinosarcoma, wherein the combination provides a synergistic effect.

According to another embodiment, the present invention provides a synergic combination comprising palbociclib and sunitinib for use in treating bones and/or soft tissues cancer, wherein the combination provides a synergistic effect. According to some embodiments, the cancer is Ewing sarcoma.

According to some embodiments, the present invention provides a synergic combination of palbociclib and sunitinib for use in treating solid cancer, wherein palbociclib is administered in a dose of from 20 to 300 mg/day and/or sunitinib is administered in the dose of from 5 to 150 mg/day. According to one embodiment, palbociclib is administered in a dose of from 20 to 300 mg/day. According to some embodiments, palbociclib is administered in the dose of 25 to 250, 30 to 225, 35 to 200, 40 to 175, 50 to 150, 55 to 130, 60 to 125, or 75 to 100 mg/day. According to another embodiment, palbociclib is administered in the dose of 25 to 150, 50 to 125 or 75 to 100 mg/day. According to another embodiment, sunitinib is administered in the dose of from 5 to 150 mg/day. According to one embodiment, sunitinib is administered in the dose of 10 to 125, 15 to 110, 20 to 100, 25 to 75, 30 to 70, 35 to 60 or 40 to 55 mg/day. According to another embodiment, sunitinib is administered in the dose of 5 to 50 mg/day, 10 to 40 mg/day or 20 to 30 mg/day. According to one embodiment, palbociclib is administered in the dose of 25 to 250 mg/day and sunitinib is administered in the dose of 10 to 125.

According to one embodiment, the combination of palbociclib and sorafenib, provides a synergistic anti-cancer effect. According to one embodiment, the cancer is not hepatocellular carcinoma.

According to another embodiment, the present invention provides a synergic combination comprising palbociclib and sorafenib for use in treating ovarian cancer, wherein the combination provides a synergistic effect.

According to another embodiment, the present invention provides a synergic combination comprising palbociclib and sorafenib for use in treating lung cancer, wherein the combination provides a synergistic effect.

According to some embodiments, the present invention provides a synergic combination of palbociclib and sorafenib for use in treating solid cancer, wherein palbociclib is administered in a dose of from 20 to 300 mg/day and/or sorafenib is administered in the dose of 100 to 950. According to one embodiment, palbociclib is administered in a dose of from 20 to 300 mg/day. According to some embodiments, palbociclib is administered in the dose of 25 to 250, 30 to 225, 35 to 200, 40 to 175, 50 to 150, 55 to 130, 60 to 125, or 75 to 100 mg/day. According to another embodiment, palbociclib is administered in the dose of 25 to 150, 50 to 125 or 75 to 100 mg/day. According to one embodiment, sorafenib is administered in the dose of 100 to 950, 150 to 900, 200 to 850, 250 or 300 to 800, 400 to 700, or 500 to 600 mg/day. According to another embodiment, sorafenib is administered in the dose of 100 to 900, 150 to 850, 200 to 800, or 300 to 700 mg/day. According to one embodiment, palbociclib is administered in the dose of 25 to 150 mg/day and sorafenib is administered in the dose of from 50 to 1200 mg/day.

According to some embodiments, the daily administered dose of at least one of the compounds of the combination is lower than the standard daily dose of said compound. According to some embodiments, the daily administered dose of at least one of the compound is lower by 5%, 10%, 15%, 20%, 25%, 30%, 35%, 40%, 45%, 50%, 55%, 60%, 65%, 70%, 75%, 80%, 85%, 90%, 95%, 97%, 99%, 99.9%, or 99.99% than the standard daily dose of said compound.

According to some embodiments, the combination is a combination of palbociclib and sunitinib, and the daily administered dose of palbociclib is 95%, 90%, 85%, 80%, 75%, 70%, 65%, 60%, 55%, 50%, 45%, 40%, 35%, 30%, 25%, 20%, 15%, or 10% of the standard daily dose of palbociclib. According to some embodiments, the daily administered dose of palbociclib is from 90% to 60% of the standard daily dose of palbociclib. According to some embodiments, the daily administered dose of palbociclib is from 80% to 40% of the standard daily dose of palbociclib. According to some embodiments, the daily administered dose of palbociclib is from 60% to 20% of the standard daily dose of palbociclib. According to some embodiments, the combination of a combination of palbociclib and sunitinib, and the daily administered dose of sunitinib is 95%, 90%, 85%, 80%, 75%, 70%, 65%, 60%, 55%, 50%, 45%, 40%, 35%, 30%, 25%, 20%, 15%, or 10% of the standard daily dose of sunitinib. According to some embodiments, the daily administered dose of sunitinib is from 90% to 60% of the standard daily dose of sunitinib. According to some embodiments, the daily administered dose of palbociclib is from 80% to 40% of the standard daily dose of sunitinib. According to some embodiments, the daily administered dose of palbociclib is from 60% to 20% of the standard daily dose of sunitinib. According to some embodiments, the daily administered dose of palbociclib is from 90% to 60% of the standard daily dose of palbociclib and the daily administered dose of sunitinib is from 90% to 60% of the standard daily dose of sunitinib.

According to some embodiments, the combination is a combination of palbociclib and sorafenib, and the daily administered dose of palbociclib is 95%, 90%, 85%, 80%, 75%, 70%, 65%, 60%, 55%, 50%, 45%, 40%, 35%, 30%, 25%, 20%, 15%, or 10% of the standard daily dose of palbociclib. According to some embodiments, the daily administered dose of palbociclib is from 90% to 60% of the standard daily dose of palbociclib. According to some embodiments, the daily administered dose of palbociclib is from 80% to 40% of the standard daily dose of palbociclib. According to some embodiments, the daily administered dose of palbociclib is from 60% to 20% of the standard daily dose of palbociclib. According to some embodiments, the combination of a combination of palbociclib and sorafenib, and the daily administered dose of sorafenib is 95%, 90%, 85%, 80%, 75%, 70%, 65%, 60%, 55%, 50%, 45%, 40%, 35%, 30%, 25%, 20%, 15%, or 10% of the standard daily dose of sorafenib. According to some embodiments, the daily administered dose of sorafenib is from 90% to 60% of the standard daily dose of sorafenib. According to some embodiments, the daily administered dose of palbociclib is from 80% to 40% of the standard daily dose of sorafenib. According to some embodiments, the daily administered dose of palbociclib is from 60% to 20% of the standard daily dose of sorafenib. According to some embodiments, the daily administered dose of palbociclib is from 90% to 60% of the standard daily dose of palbociclib and the daily administered dose of sorafenib is from 90% to 60% of the standard daily dose of sorafenib.

The synergic combination of the present invention such as a combination of palbociclib and sunitinib or palbociclib generate much less adverse effect and/or toxic effect that the known chemotaxic drugs used as a standard of care (SOC).

According to any one of the above embodiment, the combination of the present invention may be administered by any know route of administration. The term “administering” or “administration of” a compound or a combination to a subject can be carried out using one of a variety of methods known to those skilled in the art. For example, a compound, a composition or an agent can be administered, intravenously, arterially, intradermally, intramuscularly, intraperitonealy, intravenously, subcutaneously, ocularly, sublingually, orally (by ingestion), intranasally (by inhalation), intraspinally, intracerebrally, and transdermally (by absorption, e.g., through a skin duct). A compound, a composition or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to some embodiments, the combination is orally administered.

According to one embodiment, the CDK 4/6 inhibitor and the mtRTKI are administered in a sequential manner. According to another embodiment, the CDK 4/6 inhibitor and the mtRTKI are administered in a substantially simultaneous manner. The term “sequential manner” refers to an administration of two compounds at different times, and optionally in different modes of administration. The agents can be administered in a sequential manner in either order. The terms “substantially simultaneous manner” refers to administration of two compounds with only a short time interval between them. In some embodiments, the time interval is in the range of from 0.01 to 60 minutes. According to one embodiment, palbociclib and sunitinib are administered in a sequential manner. According to another embodiment, palbociclib and sunitinib are administered in substantially simultaneous manner. According to one embodiment, palbociclib and sorafenib are administered in sequential manner. According to another embodiment, palbociclib and sorafenib are administered in substantially simultaneous manner.

According to some embodiments, each of the compounds, i.e. the CDK 4/6 inhibitor and the mtRTKI, is formulated as a separate dosage form. According to other embodiments, the each compound of the combination wherein the combination is selected from palbociclib and sunitinib, palbociclib and pazopanib, abemaciclib and sorafenib, abemaciclib and pazopanib, ribociclib and sunitinib, ribociclib and sorafenib and ribociclib and pazopanib is formulated as a separate dosage form. According to some any one of the above embodiments, the dosage form is a pharmaceutical composition. According to one embodiment, each compound is formulated as a separate oral dosage form, e.g. a tablet or a capsule. According to some embodiments, each one of the separate dosage forms is administered in a route selected from oral, IV and IM route of administration.

According to another embodiment, the combination is formulated as a single dosage form. According to one embodiment, the combination selected from palbociclib and sunitinib, palbociclib and sorafenib, palbociclib and pazopanib, abemaciclib and sorafenib, abemaciclib and pazopanib, abemaciclib and sunitinib, ribociclib and sunitinib, ribociclib and sorafenib and ribociclib and pazopanib is formulated as a single dosage form. According to one embodiment, the dosage form is formulated for oral administration. According to some embodiments, the combination of palbociclib and sunitinib is formulated a single dosage form. According to some embodiments, the combination of palbociclib and sorafenib is formulated a single dosage form.

Any dosage form may be used according to the present invention. For example, the formulation of the compound or the combination may be any one selected from plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules. According to one embodiment, the dosage form is selected from the group consisting of tablets, pills, capsules, pellets, granules, powders, suspensions, dispersions, emulsions, injectable solutions, syrups, aerosols, ointments, soft and hard gelatin capsules, suppositories. According to one embodiment, the dosage form is a tablet.

According to some embodiments, the present invention provides a combination of an inhibitor of cyclin-dependent kinase 4/6 (CDK 4/6) and a multi-targeted receptor tyrosine kinase inhibitor (mtRTKI) for use in treating solid cancer, provided that the combination is not a combination selected from (i) palbociclib and sorafenib and (ii) abemaciclib and sunitinib. According to one embodiment, the CDK 4/6 inhibitor is selected from palbociclib, abemaciclib and ribociclib. According to one embodiment, the CDK 4/6 inhibitor is palbociclib. Thus in some embodiments, the present invention provides a combination of palbociclib and multi-targeted receptor tyrosine kinase inhibitor, for use in treating solid cancer, provided that mtRTKI is not sorafenib. According to another embodiment, the CDK 4/6 inhibitor is abemaciclib. Thus in some embodiments, the present invention provides a combination of palbociclib and multi-targeted receptor tyrosine kinase inhibitor, for use in treating solid cancer, provided that mtRTKI is not sunitinib. According to yet another embodiment, the CDK 4/6 inhibitor is ribociclib. Thus, in one embodiment, the present invention provides a combination of ribociclib and multi-targeted receptor tyrosine kinase inhibitor, for use in treating solid cancer. According to one embodiment, the mtRTKI is selected from sunitinib, sorafenib and pazopanib. According to one embodiment, the mtRTKI is sunitinib. Therefore, according to some embodiments, the present invention provides a combination of CDK 4/6 inhibitor and sunitinib, for use in treating solid cancer, provided that CDK 4/6 inhibitor is not abemaciclib. According to another embodiment, the mtRTKI is sorafenib. Therefore, according to some embodiments, the present invention provides a combination of CDK 4/6 inhibitor and sorafenib, for use in treating solid cancer, provided that CDK 4/6 inhibitor is not palbociclib. According to yet another embodiment, the mtRTKI is pazopanib. Therefore according to some embodiments, the present invention provides a combination of CDK 4/6 inhibitor and pazopanib, for use in treating solid cancer. All above terms and definitions apply herein as well.

According to some aspects, the present invention provides a kit comprising an inhibitor of cyclin-dependent kinase 4 and 6 (CDK 4/6), a multi-targeted receptor tyrosine kinase inhibitor (mtRTKI) and instructions for use. According to some embodiments, the present invention provides a kit comprising a pharmaceutical composition comprising an inhibitor of cyclin-dependent kinase 4 and 6 (CDK 4/6), a pharmaceutical composition comprising a multi-targeted receptor tyrosine kinase inhibitor (mtRTKI) and instructions for use. According to some embodiments, the CDK 4/6 inhibitor is selected from palbociclib, abemaciclib and ribociclib and the mtRTKI is selected from sunitinib, sorafenib and pazopanib. According to one embodiment, the kit does not comprise a combination selected from (i) palbociclib and sorafenib and (ii) abemaciclib and sunitinib. According to some embodiments, the present invention provides a kit comprising a pharmaceutical composition comprising palbociclib, a pharmaceutical composition comprising sunitinib (mtRTKI) and instructions for use. According to one embodiment, the kit comprises a pharmaceutical composition comprising from 25 to 200 mg of palbociclib and a pharmaceutical composition comprising from 5 to 75 mg of sunitinib. According to some embodiments, the present invention provides a kit comprising a pharmaceutical composition comprising palbociclib, a pharmaceutical composition comprising sorafenib (mtRTKI) and instructions for use. According to one embodiment, the kit comprises a pharmaceutical composition comprising from 25 to 200 mg of palbociclib and a pharmaceutical composition comprising from 100 to 300 mg of sorafenib. According to another embodiment, kit comprises a pharmaceutical composition comprising from 50 to 600 mg of ribociclib and a pharmaceutical composition comprising 100 to 500 mg of pazopanib. According to some embodiments, the kit comprises a pharmaceutical composition comprising from 25 to 300 mg a pharmaceutical composition comprising abemaciclib and 100 to 300 mg of sorafenib. According to some embodiments, the kit is for use in treating solid cancer. According to some embodiments, the cancer is not a hepatocellular carcinoma. According to another embodiment, the cancer is not renal cell carcinoma. According to other embodiments, the cancer is not appendix carcinoma. According to one embodiment, the cancer is selected from lymphoma, melanoma, neuroendocrine tumor, carcinoma, Ewing sarcoma, carcinosarcoma and sarcoma. According to some embodiments, the cancer is selected from lung cancer, stomach, small bowel cancer, breast cancer, ovarian cancer, bone cancer, pancreas cancer, cholangiocarcinoma, gallbladder cancer, bile duct cancer, liposarcoma, brain tumor, uterus cancer, cervical cancer, head and neck, parotid, salivary gland, adrenal cancer and colon cancer. According to some embodiments, the present invention provides a kit comprising a pharmaceutical composition comprising from 25 to 200 mg of palbociclib and a pharmaceutical composition comprising from 5 to 75 mg of sunitinib, and instructions for use in treating solid cancer. According to some embodiments, it is provided that the cancer is not a hepatocellular carcinoma. According to other embodiments, it is provided that the cancer is not renal cell carcinoma. According to one embodiment, the cancer is not appendix cancer. According to some embodiments, the present invention provides a kit comprising a pharmaceutical composition comprising from 25 to 200 mg of palbociclib and a pharmaceutical composition comprising from form 100 to 300 mg of sorafenib and instructions for use in treating cancer, provided that the cancer is not a hepatocellular carcinoma. According to some embodiments, the use provides a synergistic anticancer effect.

According to another aspect, the present invention provides a pharmaceutical composition comprising at least one inhibitor of cyclin-dependent kinase 4/6 (CDK 4/6) and at least one multi-targeted receptor tyrosine kinase inhibitor (mtRTKI), and a pharmaceutically acceptable excipient. According to one embodiment, the CDK 4/6 inhibitor is selected from palbociclib, abemaciclib and ribociclib. According to another embodiment, the mtRTKI is selected from sunitinib, sorafenib and pazopanib. According to a further embodiment, the present invention provides a pharmaceutical composition comprising an inhibitor of CDK 4/6 and an mtRTKI, wherein the CDK 4/6 inhibitor is selected from palbociclib, abemaciclib and ribociclib and the mtRTKI is selected from sunitinib, sorafenib, and pazopanib.

The term “pharmaceutical composition” as used herein refers to a composition comprising the compound or a combination of compounds of the present invention formulated together with one or more pharmaceutically acceptable carriers.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain solid carriers or excipients such as, for example, lactose, starch or talcum or liquid carriers such as, for example, water, fatty oils or liquid paraffin.

These formulations can be produced by known methods using conventional solid carriers or excipients such as, for example, lactose, starch or talcum or liquid carriers such as, for example, water, fatty oils or liquid paraffin. Other carriers or excipients which may be used include, but are not limited to, materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminium silicates; and amino acids having from 2 to 12 carbon atoms and derivatives thereof such as, but not limited to, glycine, L-alanine, L-aspartic acid, L-glutamic acid, L-hydroxyproline, L-isoleucine, L-leucine and L-phenylalanine. Each possibility represents a separate embodiment of the present invention.

Auxiliary components such as tablet disintegrants, solubilisers, preservatives, antioxidants, surfactants, viscosity enhancers, coloring agents, flavoring agents, pH modifiers, sweeteners or taste-masking agents may also be incorporated into the composition. Suitable coloring agents include, but are not limited to, red, black and yellow iron oxides and FD & C dyes such as FD & C blue No. 2 and FD & C red No. 40 available from Ellis & Everard. Suitable flavoring agents include, but are not limited to, mint, raspberry, liquorice, orange, lemon, grapefruit, caramel, vanilla, cherry and grape flavors and any combinations thereof. Suitable pH modifiers include, but are not limited to, citric acid, tartaric acid, phosphoric acid, hydrochloric acid and maleic acid. Suitable sweeteners include, but are not limited to, aspartame, acesulfame K and thaumatin. Suitable taste-masking agents include, but are not limited to, sodium bicarbonate, ion-exchange resins; cyclodextrin inclusion compounds, adsorbates or microencapsulated actives. Each possibility represents a separate embodiment of the present invention.

According to some embodiments, the pharmaceutical composition may be administered via any known route of administration. According to one embodiment, the pharmaceutical composition is administered via a route selected from the group consisting of oral, rectal, intramuscular, subcutaneous, intravenous, intraperitoneal, intranasal, intraarterial, intravesicle, intraocular, transdermal and topical. Thus, the composition of the present invention is formulated for administered via a route selected from the group consisting of oral, rectal, intramuscular, subcutaneous, intravenous, intraperitoneal, intranasal, intraarterial, intravesicle, intraocular, transdermal and topical.

According to certain embodiments, the compounds and compositions of the present invention are particularly suitable for oral administration. It is contemplated that by orally administering the compounds and compositions of the present invention, a systemic effect can be achieved.

According to some embodiments, the present invention provides a pharmaceutical composition comprising a combination selected from palbociclib and sunitinib; palbociclib and sorafenib, palbociclib and pazopanib; abemaciclib and sorafenib; abemaciclib and pazopanib; ribociclib and sunitinib; abemaciclib and sunitinib, ribociclib and sorafenib; and ribociclib and pazopanib. According to one embodiment, the pharmaceutical composition is formulated for oral administration. According to a certain embodiment, the pharmaceutical composition is in a form of a tablet or a capsule.

According to some embodiments, the present invention provides a pharmaceutical composition comprising palbociclib and sunitinib. According to one embodiment, the pharmaceutical composition comprises from 25 to 250 mg of palbociclib and from 5 to 75 mg of sunitinib. According to one embodiment, the pharmaceutical composition comprises from 25 to 250, 30 to 225, 35 to 200, 40 to 175, 50 to 150, 55 to 130, 60 to 125, or 75 to 100 mg of palbociclib. According to another embodiment, the pharmaceutical composition comprises from 10 to 70, 15 to 65, 20 to 60, 20 to 60, 25 to 55, 30 to 50 or 35 to 45 mg of sunitinib. According to one embodiment, the pharmaceutical composition comprises from 25 to 250, 30 to 225, 35 to 200, 40 to 175, 50 to 150, 55 to 130, 60 to 125, or 75 to 100 mg of palbociclib and from 10 to 70, 15 to 65, 20 to 60, 20 to 60, 25 to 55, 30 to 50 or 35 to 45 mg of sunitinib.

According to some embodiments, the present invention provides a pharmaceutical composition comprising palbociclib and sorafenib. According to one embodiment, the pharmaceutical composition comprises from 25 to 200 mg of palbociclib and from 100 to 300 mg of sorafenib. According to one embodiment, the pharmaceutical composition comprises from 25 to 250, 30 to 225, 35 to 200, 40 to 175, 50 to 150, 55 to 130, 60 to 125, or 75 to 100 mg of palbociclib. According to another embodiment, the pharmaceutical composition comprises from 110 to 290, 120 to 280, 130 to 260, 150 to 240, 180 to 220, 190 to 210, 100 to 200 or 200 to 300 mg of sorafenib. According to one embodiment, the pharmaceutical composition comprises from 25 to 250, 30 to 225, 35 to 200, 40 to 175, 50 to 150, 55 to 130, 60 to 125, or 75 to 100 mg of palbociclib and from 110 to 290, 120 to 280, 130 to 260, 150 to 240, 180 to 220, 190 to 210, 100 to 200 or 200 to 300 mg of sorafenib.

According to some embodiments, the present invention provides a pharmaceutical composition comprising ribociclib and pazopanib. According to one embodiment, the pharmaceutical composition comprises from 50 to 600 mg of ribociclib and 100 to 500 mg of pazopanib. According to one embodiment, the pharmaceutical composition comprises from 50 to 600 mg, 75 to 575, 100 to 550, 125 to 525, 150 to 500, 200 to 450, 250 to 400 or 300 to 350 of ribociclib. According to another embodiment, the pharmaceutical composition comprises from 150 to 450, from 200 to 400, from 250 to 350 mg of pazopanib.

According to one embodiment, the present invention provides a pharmaceutical composition comprising abemaciclib and sorafenib. According to one embodiment, the pharmaceutical composition comprises from 25 to 300 mg of abemaciclib and 100 to 300 mg of sorafenib. According to another embodiment, the pharmaceutical composition comprises 110 to 290, 120 to 280, 130 to 260, 150 to 240, 180 to 220, 190 to 210, 100 to 200 or 200 to 300 mg of sorafenib. According to another embodiment, the pharmaceutical composition comprises 30 to 290, 50 to 280, 100 to 260, 150 to 240, 180 to 220, 190 to 210, 100 to 200 or 200 to 300 mg of abemaciclib

According to any one of the above embodiments, the pharmaceutical composition is a synergic composition. Thus, the pharmaceutical composition of the present invention is a synergic pharmaceutical composition.

According to any one of the above embodiments, the pharmaceutical composition is for use in treating solid cancer. According to some embodiments, the cancer is not hepatocellular carcinoma and/or not renal cell carcinoma. According to one embodiment, cancer is selected from lymphoma, melanoma, neuroendocrine tumor, carcinoma, Ewing sarcoma, carcinosarcoma and sarcoma. According to another embodiment, the cancer is selected from lung, stomach, breast, ovarian and colon cancer, neuroendocrine cancer, pancreas cancer, cholangiocarcinoma, bone cancer, gallbladder cancer, bile duct cancer, liposarcoma, and adrenal cancer.

According to one embodiment, the present invention provides a pharmaceutical composition comprising palbociclib and sunitinib, for use for use in treating solid cancer. According to one embodiment the present invention provides a pharmaceutical composition comprising palbociclib and sorafenib for use in treating cancer. According to some embodiments, the cancer is not hepatocellular carcinoma. According to some embodiments, the cancer is not appendix cancer. According to some embodiments, the pharmaceutical composition provides a synergistic anticancer effect. According to some embodiments, the synergistic pharmaceutical composition is for use in treating cancer selected from lung cancer, stomach cancer, breast cancer, ovarian cancer, neuroendocrine cancer, pancreas cancer, bones cancer and cholangiocarcinoma. According to one embodiment, the breast cancer is 5FU resistant and/or palbociclib resistant breast cancer. According to some embodiments, the cancer is characterized by KRAS mutation. According to one embodiment, KRAS mutation is G12V mutation. According to other embodiments, the cancer is characterized by HER2 negative mutation. According to some embodiments, the cancer is estrogen receptor positive (ER+). According to yet another embodiment, the cancer is characterized by presence BRCA1 gene. According to yet another embodiment, the cancer expresses wild-type RAS protein.

According to another aspect, the present invention provides a method of treating solid cancer in a subject in need thereof comprising co-administering an inhibitor of cyclin-dependent kinase 4/6 (CDK 4/6) selected from palbociclib, abemaciclib and ribociclib and multi-targeted receptor tyrosine kinase inhibitor (mtRTKI) selected from sunitinib, sorafenib and pazopanib. According to some embodiments, it is provided that the cancer is not hepatocellular carcinoma and/or not a renal cell carcinoma. According to some embodiments, treating provides a synergistic effect. Thus, according to one embodiment, the present intention provides a method of treating solid cancer in a subject in need thereof comprising administering palbociclib and sunitinib, wherein the method provides a synergic anticancer effect, provided that the cancer is not hepatocellular carcinoma or appendix cancer. According to another embodiment, the present intention provides a method of treating solid cancer in a subject in need thereof comprising administering palbociclib and sorafenib, wherein the method provides a synergic anticancer effect, provided that the cancer is not hepatocellular carcinoma. According to one embodiment, cancer is selected from lymphoma, melanoma, neuroendocrine tumor, carcinoma, Ewing sarcoma, carcinosarcoma and sarcoma. According to another embodiment, the cancer is selected from lung, stomach, breast, ovarian and colon cancer, neuroendocrine cancer, pancreas cancer, cholangiocarcinoma, bone cancer, liposarcoma, and adrenal cancer.

According to another aspect, the present invention provides a method of treating solid cancer in a subject in need thereof comprising a pharmaceutical composition comprising co-an inhibitor of cyclin-dependent kinase 4/6 (CDK 4/6) and multi-targeted receptor tyrosine kinase inhibitor (mtRTKI) of the present invention.

According to another embodiment, the present invention provides a method of treating solid cancer comprising co-administering an inhibitor of cyclin-dependent kinase 4/6 (CDK 4/6) and multi-targeted receptor tyrosine kinase inhibitor (mtRTKI), provided that the combination is not a combination selected from (i) palbociclib and sorafenib and (ii) abemaciclib and sunitinib. According to one embodiment, the CDK 4/6 inhibitor is selected from palbociclib, abemaciclib and ribociclib and the mtRTKI is selected from sunitinib, sorafenib and pazopanib.

According to another aspect, the present invention provides use of CDK 4/6 selected from palbociclib, abemaciclib and ribociclib and mtRTKI selected from sunitinib, sorafenib and pazopanib for preparation of a medicament for treating solid cancer. According to some embodiments, cancer is not selected from hepatocellular carcinoma or renal cell carcinoma. According to one embodiment, the present invention provides use of palbociclib and sunitinib, for preparation of a medicament for treating solid cancer. According to another embodiment, the present invention provides use of palbociclib and sorafenib, for preparation of a medicament for treating solid cancer.

The terms “co-administration” as used herein have the meaning of administering two or more compound in a regimen selected from a single combined composition, separate individual compositions administered substantially at the same time, and separate individual compositions administered under separate schedules and include treatment regimens in which the compound are not necessarily administered by the same route of administration or at the same time. According to some embodiments, the term “co-administration” encompasses administration of a first and second compound in an essentially simultaneous manner, such as in a single dosage form, e.g., a capsule or tablet having a fixed ratio of first and second amounts, or in multiple dosage forms for each. The agents can be administered in a sequential manner in either order.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Having now generally described the invention, the same will be more readily understood through reference to the following examples, which are provided by way of illustration and are not intended to be limiting of the present invention.

### EXAMPLES

Materials and Methods

Cancer specimens were obtained from core needle biopsy, tumor resection, bone-marrow (BM) aspiration and biopsies, acytes or pleural effusion of patients diagnosed and treated at the Davidoff center, Rabin medical center of Israel.

All mice were maintained and treated in accordance with the Rabin medical center guide for the care and use of experimental animals with approval from the RMC Institutional Animal Care and Use Committee.

Tumor materials were placed in cold DMEM medium supplemented with 10% FBS and 1:100 penicillin/streptomycin antibiotics and maintained on ice until processing. Within 0.5-2 hours, tumor fragments were cut into small pieces (approximately 2-3 mm) using sterile surgical instruments. Several pieces were used for implantation and the remaining pieces were preserved in 10% DMSO/90% FCS freezing medium, snap-frozen in liquid nitrogen and another piece was formalin-fixed for later histological examination. Typically, several implantations were carried out: subcutaneously on the flanks, intraperitoneal implantation, and implantation directly into the mammary of recipient 5-8 weeks old female immunodeficient mice NRG or NSG mice (Jackson Laboratories: NSG-NOD.Cg-PrkdcscidIl2rgtm1Wjl/SzJl strain and NRG is NOD.Cg-Rag1tm1Mom Il2rgtm1Wjl/SzJ strain). For samples obtained from breast cancer patients, mice were also supplemented with 17b-estradiol (Tocris, Cas #50-28-2) using slow release by osmotic pumps implanted subcutaneous on the back (28-day release, 1.08 mg/pellet, Alzet). Before implantation, the tumor fragments were coated with Cultrex Basement membrane matrix, type3 (Trevigen). For samples from either pleural effusion, ascites or BM: acytes/pleural fluid was centrifuged at 1500 RPM for 4 min, washed with PBS and cells were counted. BM samples: MNCs were isolated using Ficoll (Sigma), and cells were counted. About 2-3 million cells resuspended in a volume of 0.05-0.1 ml Cultrex:PBS (1:1) were injected SC, IP, or IV. In some cases of BM samples, injection was done from the total BM aspiration without any manipulation. Surgery was performed under sterile conditions in a laminar flow cabinet using sterilized surgical instruments. Mice were kept under pathogen-free conditions and received sterilized food and water ad libitum.

Tumor Growth

Mice were weighted and inspected 1-2/week for assessment of general condition and PDX development was assessed by palpation of the site of implantation and measured in two dimensions by electronic caliper. Tumor volume was determined by the ellipsoidal formula (width×width×length)/2. Once tumors reached 1-1.5 cm in diameter, mice were euthanized and tumors were harvested. Tumor tissue was directly passaged into further generation or for storage. Pathological assessment of tumors: Paraffin blocks (FFPE) were prepared from tumor sections preserved in 4% PFA and slides were stained with Hematoxilin & Eosin (H&E) solution for initial histopathological evaluation. Histopathological examination confirmed their human origin and their morphological similarity to the corresponding engrafted tumor.

Drug Efficacy Experiments

Fresh tumors were excised from mice, dissociated by GentleMACS and implanted by subcutaneous injection into the neck of NRG mice. Mice were used at age of 7-12 weeks, gender of the mice were in accordance with patient's gender. When tumors reached a size of 60-200 mm³, mice were assigned to the various treatment groups based on the tumor size, tumor growth rate and body weight (BW), creating groups with similar average of these parameters. Drugs dosing were initiated at the same day of randomization, denoted as Day 0. Mice were treated with vehicle, or with drugs as described in the experiments below. All doses were delivered by oral gavage in a total volume of 100 μL/28 gr. Drugs were administered daily for 5 days/week

Tumor volume and BW were measured twice a week throughout the treatment period. When tumor volume reached 1500 mm³, mice were euthanized according to IACUC approved protocol. The tumors were surgically removed and stored (fixed at 4% PFA followed by FFPE, and freezed in liquid-nitrogen).

Survival curves were calculated using Kaplan-Meier approach, and comparison the survival curves was done using log rank test (Kuhfeld and So, “Creating and Customizing the Kaplan-Meier Survival Plot in PROC LIFETEST”, SAS Global Forum 2013, SAS Institute Inc.)

Statistical Analysis

Statistics calculations were performed by a third independent party. All measured variables and derived parameters were tabulated by descriptive statistics.

For continuous variables summary tables are provided giving sample size, arithmetic mean, standard deviation, coefficient of variation, median, minimum and maximum.

The experiments were performed using constant drug doses and the comparison between the groups was done the last day in which all the treatment groups of Drug A, Drug B, combination of A+B were available.

Due to experimental considerations, for most experiments were performed for a single dose for each one of the compounds. As a results, a direct method to determine synergy was applied. This method includes in comparing the drugs combination (A+B) effect to each drug effect separately (A, B).

The non-parametric Wilcoxon-Mann-Whitney Rank sum test for independent samples was applied for testing the statistical significance difference between treatment groups for the primary endpoint (tumor volume) by experiment and time. P-value of 0.05 or lower is considered to be statistically significant.

To prove the synergic effect as a first the superiority of the drugs combination over each drug effect must be statistically significant.

Then, if there is no statistically significant difference between effects of drugs A and B, an average of the two effect is calculated to obtain a more precise estimate of the effect (second step). If effects of A and B are different, the average has no statistical value.

The superiority of the combination over the average effect must be statistically significant, provided that the effect of drugs A and B are not statistically different.

If the effect of the combination is higher than the effect of each one of the separate treatments with statistical significance and the effect of the combination is higher than the average effect of the two treatments with statistical significance (provided that there is no statistically significant difference between effects of two drugs) then synergy is proved. Otherwise, additive effect is concluded.

Survival Analysis using Kaplan-Meier survival function curve was applied for testing the statistical significance of the difference in overall survival between treatment groups by experiment and for all experiments pooled together. The Log-Rank test was used for treatment comparison

All tests were two-tailed, and p value of 5% or less was considered statistically significant.

### Example 1A. Efficacy of the Palbociclib+Sunitinib Combo Treatment of Colon Cancer (RA-300)

Colon cancer tissue (adenocarcinoma comprising KRAS mutation G12V), obtained by an ultrasound directed liver biopsy from a liver metastatic lesion, was collected from the liver, grown in NRG mice, collected dissociated and implanted in new NRG mice and when reached a size of 60-110 mm³, mice were assigned to treatment groups as described in Table 1.

TABLE 1
Study Design

| | | # of treatments | Treatment | # of mice |
|---|---|---|---|---|
| Group # | Tested drugs | per week | method | per group |
| 1 | control (vehicle) | 5 | PO | 5 |
| 2 | palbociclib 100 mg/kg | 5 | PO | 6 |
| 3 | sunitinib 50 mg/kg | 5 | PO | 5 |
| 4 | palbociclib 100 mg/kg + | 5 | PO | 6 |
| | sunitinib 50 mg/kg | | | |
| 5 | gemcitabine 25 mg/kg | 2 | IP | 5 |

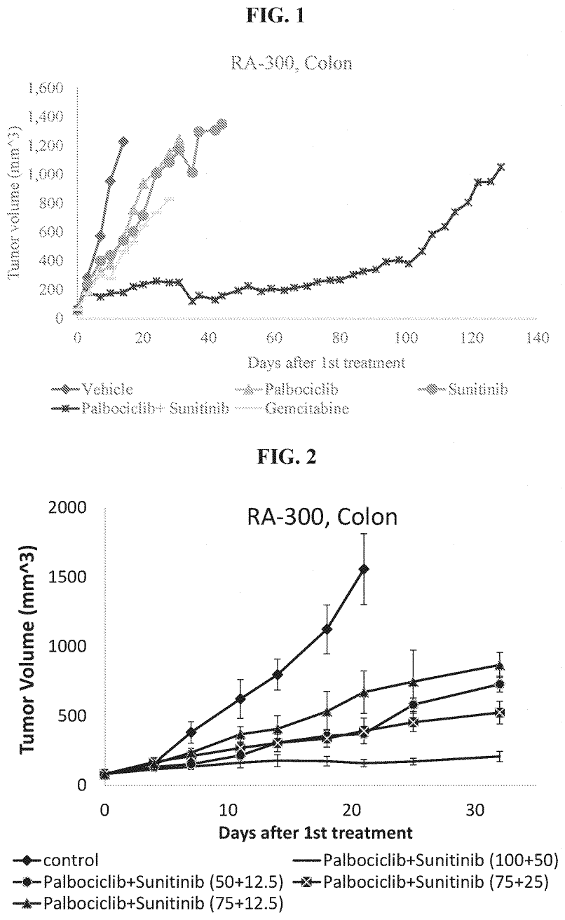

The experiment lasted 129 days. Vehicle was used as a negative control whereas gemcitabine (known anticancer drug) as aSOC (standard of care) treatment. The results, showing effect of palbociclib, sunitinib and their combination on tumor volume size, are presented in FIG. 1. It can be clearly seen from FIG. 1 that whereas cancer in the presence each of the anticancer drugs alone quickly progressed, concomitant administration of palbociclib and sunitinib significantly and effectively inhibited growth of the tumor for more than 70 days. It seems that after about 60 days of tumor growth inhibition in the combo group, tumors start to develop resistance to therapy and re-grow. After >100 days of treatment in the combo group, 2 mice developed ulcers. It should be also emphasized that the combination was much more effective than the standard treatment with gemcitabine. The synergic effect of palbociclib and sunitinib combination is statistically significant as follows from P-values of Wilcoxon Test presented in Table 2 and from log-rank test of overall survival testing combination palbociclib and sunitinib vs each one of these drugs alone.

TABLE 2
Wilcoxon Test P-values

| | | P-value, Wilcoxon Test |
|---|---|---|
| Treatment | Day | (Two-sided) |
| Palbociclib Vs combo | 31 | 0.0081 |
| Sunitinib Vs combo | 31 | 0.0122 |
| Mean Palbociclib Sunitinib Vs | 31 | 0.0081 |
| combo | | |

TABLE 3
Log-rank values

| Treatment | Log-Rank |
|---|---|
| Palbociclib Vs combo | 0.0099 |
| Sunitinib Vs combo | 0.0494 |

It can be clearly seen from that experiment that the combination of palbociclib+sunitinib has a synergetic inhibitory effect compared to each one of palbociclib or sunitinib alone in treatment colon cancer.

Further the influence of drugs doses on tumor volume was tested for 32 days. Mice were allocated to treatment as defined in Table 3.

TABLE 3
Study Design

| | | # of treatments | Treatment | # of mice |
|---|---|---|---|---|
| Group # | Tested drugs | per week | method | per group |
| 1 | Control (vehicle) | 5 | PO | 5 |
| 2 | palbociclib 100 mg/kg + | 5 | PO | 6 |
| | sunitinib 50 mg/kg | | | |
| 3 | palbociclib 50 mg/kg + | 5 | PO | 5 |
| | sunitinib 12.5 mg/kg | | | |
| 4 | palbociclib 75 mg/kg + | 5 | PO | 6 |
| | sunitinib 25 mg/kg | | | |
| 5 | palbociclib 75 mg/kg + | 5 | IP | 6 |
| | sunitinib 12.5 mg/kg | | | |

The result are presented in FIG. 2. As can clearly be seen from the figure, the response of colon tumor to the treatment of the combination of palbociclib and sunitinib is dose related. Administrating of 2-fold lower doses of both drugs was shown to sinergically suppress tumor growth in a dose-response manner.

### Example 1B. Efficacy of the Palbociclib+Sunitinib Combo Treatment of Colon Cancer (RA-419)

In an additional experimental arrangement, colon tumor (well differentiated adenocarcinoma) was grown in NRG mice, collected dissociated and implanted in new NRG mice and when reached of 60-90 mm³, mice were assigned to treatment groups as described in Table 5.

TABLE 5
Study Design

| | | # of treatments | Treatment | # of mice |
|---|---|---|---|---|
| Group # | Tested drugs | per week | method | per group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | 5-FU 30 mg/kg + | 2 | IP | 5 |
| | Irinotecan 20 mg/kg | | | |

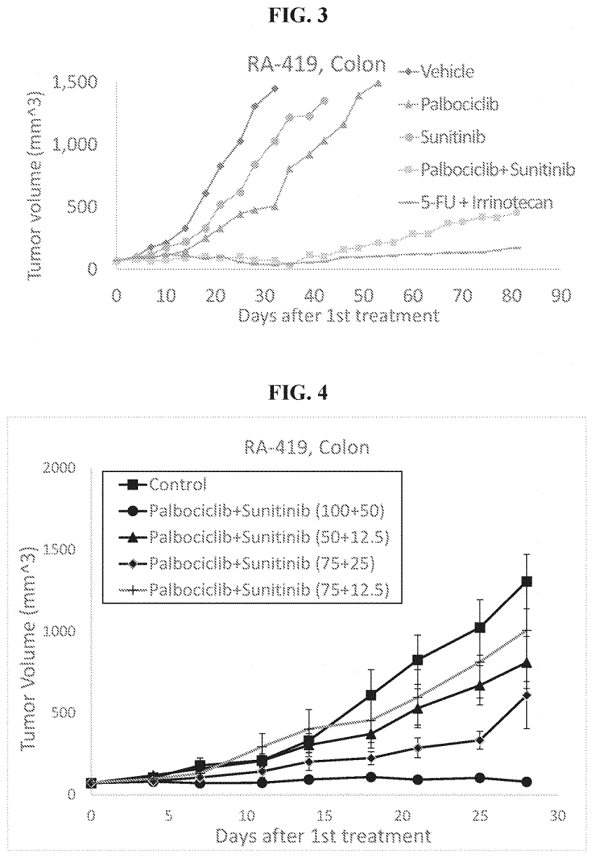

The experiment lasted 81 days. Vehicle used as a negative control whereas administration of 5-FU+Irinotecan as SOC treatment. The results showing effect of palbociclib, sunitinib and their combination on tumor volume size are presented in FIG. 3. Table 6 summarizes the P-values of Wilcoxon Test. Within the arrangement of the experiment, it was also possible to calculate Log-rank values presented in Table 7. FIG. 3 shows that palbociclib alone attenuate tumor growth and sunitinib treatment has little or almost no effect on tumor volume. It can be clearly seen from FIG. 3 that whereas cancer in the presence of each one of the anticancer drugs alone quickly progressed, concomitant administration of palbociclib and sunitinib significantly and effectively inhibited growth of the tumor for more than 80 days. It seems that after about 60 days of tumor growth inhibition in the combo group, tumors start to develop resistance to therapy and re-grow. It should be also seen that the combination has similar effect as the standard treatment with 5-FU+Irinotecan.

TABLE 6
Wilcoxon Test P-values

| Treatment | Day | P-value, Wilcoxon Test (Two-sided) |
|---|---|---|
| palbociclib Vs combo | 42 | 0.0122 |
| sunitinib Vs combo | 42 | 0.0122 |
| Mean palbociclib | 42 | 0.0122 |
| sunitinib Vs combo | | |

TABLE 7
Log-rank values.

| Treatment | Log-Rank |
|---|---|
| palbociclib Vs combo | 0.0144 |
| sunitinib Vs combo | 0.0345 |

All these data shows that the combination of palbociclib with sunitinib synergically inhibits growth of colon cancer tumor.

Influence of doses of palbociclib and sunitinib in combination on tumor volume was tested. The experiment was performed for 28 days, and the mice were allocated according to Table 8. The results are presented in FIG. 4.

TABLE 8
Study Design

| | | # of treatments | Treatment | # of mice |
|---|---|---|---|---|
| Group # | Tested drugs | per week | method | per group |
| 1 | Control (vehicle) | 5 | PO | 5 |
| 2 | Palbociclib 100 mg/kg + | 5 | PO | 5 |
| | Sunitinib 50 mg/kg | | | |
| 3 | Palbociclib 50 mg/kg + | 5 | PO | 5 |
| | Sunitinib 12.5 mg/kg | | | |
| 4 | Palbociclib 75 mg/kg + | 5 | PO | 5 |
| | Sunitinib 25 mg/kg | | | |
| 5 | Palbociclib 75 mg/kg + | 5 | PO | 5 |
| | Sunitinib 12.5 mg/kg | | | |

A clear dose dependent response was observed for the combination. Administrating of 2-fold lower doses of both drugs was shown to sinergically suppress tumor growth in a dose-response manner.

### Example C. Efficacy of the Palbociclib+Sunitinib Combo Treatment of Colon Cancer (RA-397)

In a further experimental arrangement, colon tumor (metastatic adenocarcinoma with wild-type RAS) was grown in NRG mice, collected dissociated and implanted in new NRG mice and when reached a size of 60-170 mm³, mice were assigned to treatment groups as described in Table 9.

TABLE 9
Study Design

| | | # of treatments | Treatment | # of mice |
|---|---|---|---|---|
| Group # | Tested drugs | per week | method | per group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | Folfox (leucovorin | 1 | IP | 6 |
| | 70 mg/kg + oxaliplatin | | | |
| | 4 mg/kg + 5FU 30 mg/kg) | | | |
| | Avastin 5 mg/kg | 2 | | |

The experiment lasted 67 days. Vehicle used as a negative control whereas administration of 5-folfox+avasting as SOC treatment. The results showing effect of palbociclib, sunitinib and their combination on tumor volume size are presented in FIG. 5. Table 10 summarizes the P-values of Wilcoxon Test. FIG. 5 shows that palbociclib alone attenuate tumor growth and sunitinib treatment has only moderate effect on tumor volume. It can be clearly seen from FIG. 5 that whereas cancer in the presence of each one of the anticancer drugs alone keep progressing, concomitant administration of palbociclib and sunitinib significantly and effectively inhibited growth of the tumor for more than 60 days. Moreover, while at day 52 the tumor develops resistance to polbociclib (at this point tumor in mice treated with sunitinib reached maximal size defined for euthanization of mice), the combo treatment successfully inhibited the progression of cancer. This clearly indicates for the synergy between palbociclib and sunitinib. In addition, the combination had a long lasting effect of tumor inhibition, similar to the standard treatment. However, the combination was significantly safer to the mice than the SOC treatment. On day 18, 2 out of 5 mice in the SOC group were excluded from the experiment due to toxicity with severe clinical signs (weakness and loss of weight), compare to the combination group in which all 5 mice were treated without any toxicity signs until day 67.

TABLE 10
Wilcoxon Test P-values

| Treatment | Day | P-value, Wilcoxon Test (Two-sided) |
|---|---|---|
| Palbociclib Vs combo | 49 | 0.0216 |
| Sunitinib Vs combo | 49 | 0.0216 |
| Mean Palbociclib | 49 | 0.0122 |
| Sunitinib Vs combo | | |

### Example 2. Efficacy of the Palbociclib+Sunitinib Combo Treatment of Stomach Cancer (RA-346)

Stomach cancer tissue (poorly differentiated stomach adenocarcinoma (HER2 negative)), obtained by a biopsy from the liver was grown in NRG mice, and further collected, dissociated and implanted in new NRG mice for drug efficacy study. When tumors reached a size of 60-160 mm³, mice were assigned to treatment groups based on the tumor size, tumor growth rate and body weight (BW), creating groups with similar average of these parameters (see Table 11). Drugs dosing were initiated at the same day of randomization, denoted as Day 0.

TABLE 11
Study Design

| | | # of treatments | Treatment | # of mice |
|---|---|---|---|---|
| Group # | Tested drugs | per week | method | per group |
| 1 | Control (vehicle) | 5 | PO | 16 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 6 |
| | Sunitinib 50 mg/kg | | | |
| 5 | Ribociclib 100 mg/kg | 5 | PO | 4 |
| 6 | Pazopanib 100 mg/kg | 5 | PO | 4 |
| 7 | Ribociclib 100 mg/kg + | | PO | 5 |
| | Pazopanib 100 mg/kg | | | |
| 8 | Abemaciclib 100 mg/kg | 5 | PO | 4 |
| 9 | Sorafenib 20 mg/kg | 5 | PO | 4 |
| 10 | Abemaciclib 100 mg/kg + | | PO | 5 |
| | Sorafenib 20 mg/kg | | | |
| 11 | Oxaliplatin 4 mg/kg + | 2 | IP | 5 |
| | 5-FU 30 mg/kg | | | |

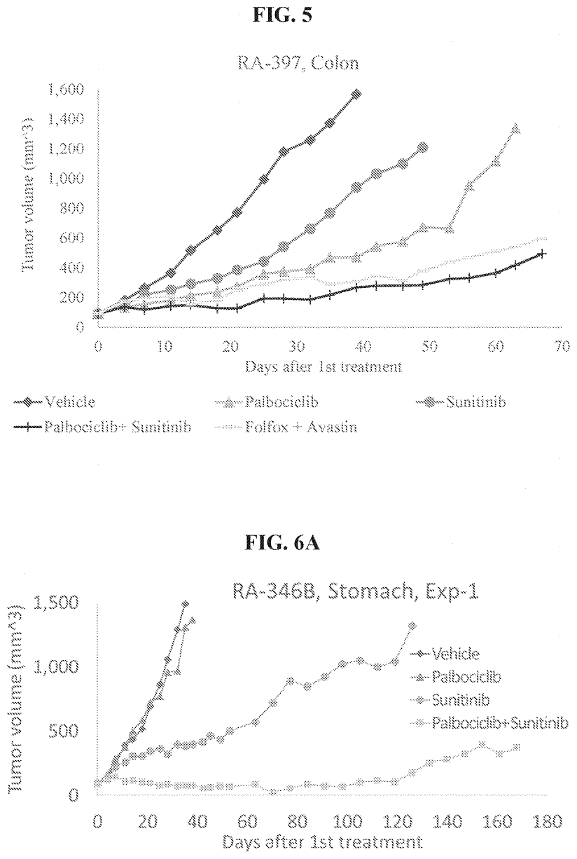

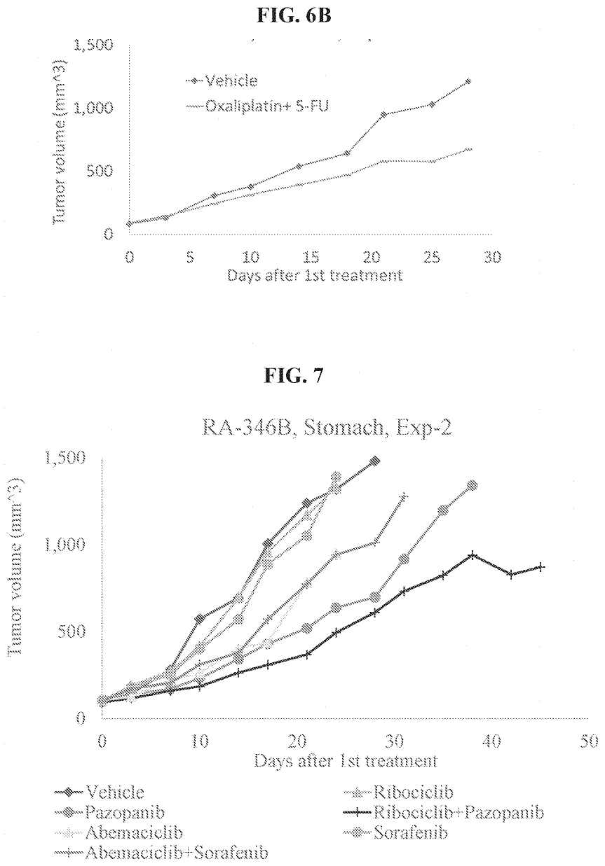

The results are presented on FIGS. 6-7

It can be clear from FIG. 6A that a combination of palbociclib and sunitinib provides a much stronger effect than it could be expected from a combination of effects of each one of these drugs. Moreover, the combination provided a long lasting effect of a complete inhibition of tumor development until day 120, and inhibiting tumor growth for additional 40 days (until the end of the experiment). At day 126, two mice show complete regression and one mice has stable disease (80 mm³). After −60 days of tumor growth inhibition in the combo group, tumors start to develop resistance to therapy and re-grow. After >100 days of treatment in the combo group, 2 mice developed ulcers (in the tumors) and removed from the study. It can be seen when expecting FIGS. 6A and 6B that the treatment with combo was much more efficient that the standard treatment with oxaliplatin+5FU.

FIG. 7 shows that not all combinations of drugs were as effective as a combination of pabociclib and sunitinib. For example a combination of robociclib and pazopanib (referred as combo 2) was not more effective than pazopanib alone. A combination of abemaciclib and sorafenic (referred as combo 3) was as effective as abemaciclib alone. As already discussed, effect of a combinatory treatment cannot be predicted and moreover, one can not predict whether a combination will have a synergic effect, as in case of a combination of palbociclib and sunitinib.

These results are statistically significant, as can be learnt from Table 12, showing P-values of Wilcoxon Test for all combinations. The statistical analysis support the conclusion that the combination of palbociclib and sunitinib are synergistic in treatment of stomach cancer, it also shows that no synergy can be seen for combo 2 and 3.

TABLE 12
Wilcoxon Test P-values

| | | P-value, Wilcoxon Test (Two- |
|---|---|---|
| Treatment | Day | sided) |
| Palbociclib Vs combo | 38 | 0.0081 |
| Sunitinib Vs combo | 38 | 0.0081 |
| Mean Palbociclib Sunitinib | 38 | 0.0081 |
| Vs combo | | |
| Sunitinib Vs combo | 126 | 0.0200 |
| Ribociclib Vs combo 2 | 24 | 0.0200 |
| Pazopanib Vs combo 2 | 24 | 0.3913 |
| Mean Ribociclib | 24 | 0.0662 |
| Pazopanib Vs combo 2 | | |
| Abemaciclib Vs combo 3 | 24 | 1.0000 |
| Sorafenib Vs combo 3 | 24 | 0.2703 |
| Mean Abemaciclib | 24 | 0.7133 |
| Sorafenib Vs combo 3 | | |

Summarizing all said above one can conclude that a combination of palbociclib+sunitinib has a synergetic effect in comparison to effect expected from administration of these drugs together in treatment of stomach cancer. Such a synergic effect was not seen for a combination of ribociclib and pazopanib and not for a combination of abemaciclib+sorafenib.

### Example 3A. Efficacy of the Palbociclib+Sunitinib Combo Treatment of Breast Cancer (RA-334)

Invasive ductal carcinoma (ER positive) that was clinically shown to be resistant to palbociclib was grown in NRG mice, and further collected, dissociated and implanted in new NRG mice for drug efficacy study. When tumors reached a size of 60-180 mm³, mice were assigned to treatment groups based on the tumor size, tumor growth rate and body weight (BW), creating groups with similar average of these parameters (Table 13). Drugs dosing were initiated at the same day of randomization, denoted as Day 0. Tumor volume was measured.

TABLE 13
Study design

| | | # of treatments | Treatment | # of mice |
|---|---|---|---|---|
| Group # | Tested drugs | per week | method | per group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 6 |
| | Sunitinib 50 mg/kg | | | |
| 5 | Ribociclib 100 mg/kg | 5 | PO | 5 |
| 6 | Pazopanib 100 mg/kg | 5 | PO | 5 |
| 7 | Ribociclib 100 mg/kg + | 5 | PO | 5 |
| | Pazopanib 100 mg/kg | | | |

The results are presented in FIG. 8. It can be seen that each one of used drugs, i.e. palbociclib, sunitinib, ribociclib and pazopanib had weak to mild effect on tumor size. Whereas combination of ribociclib and pazopanib showed an additive effect, the combination of palbociclib and sunitinib showed a synergic effect on breast cancer. The synergic effect is statistically significant as follows from P-values of Wilcoxon Test (Table 14) and further supported by log-rank values of survival test (Table 15).

TABLE 14
Wilcoxon Test P-values

| | | P-value, Wilcoxon |
|---|---|---|
| Treatment | Day | Test (Two-sided) |
| Palbociclib Vs combo | 11 | 0.0137 |
| Sunitinib Vs combo | 11 | 0.0137 |
| Mean Palbociclib | 11 | 0.0137 |
| Sunitinib Vs combo | | |
| Ribociclib Vs combo | 11 | 0.0122 |
| Pazopanib Vs combo | 11 | 0.1779 |
| Mean Ribociclib | 11 | 0.0601 |
| Pazopanib Vs combo | | |

TABLE 15

| Treatment | Log-Rank |
|---|---|
| Palbociclib Vs combo | 0.0088 |
| Sunitinib Vs combo | 0.0091 |

### Example 3B. Efficacy of the Palbociclib+Sunitinib Combo Treatment of Breast Cancer

Breast cancer cells (The cancer was clinically shown to be resistant to palbociclib (data not shown) were collected by bone marrow aspiration and were grown in immunocompromised mice. Then the grown tumor mass was collected, dissociated and implanted subcutaneously to NRG mice necks as described in material and methods. First, the mice were treated with fluorouracil (5FU) (30 mg/kg, IP 2/week), with variable attenuation of tumor growth between mice. One of the tumors was taken for further implantation to set experiment with combination of palbociclib+sunitinib. The implanted mice were treated orally with a combination of palbociclib and sunitinib (6 mice) or with a control (6 mice) 5 times/week for 20 days.

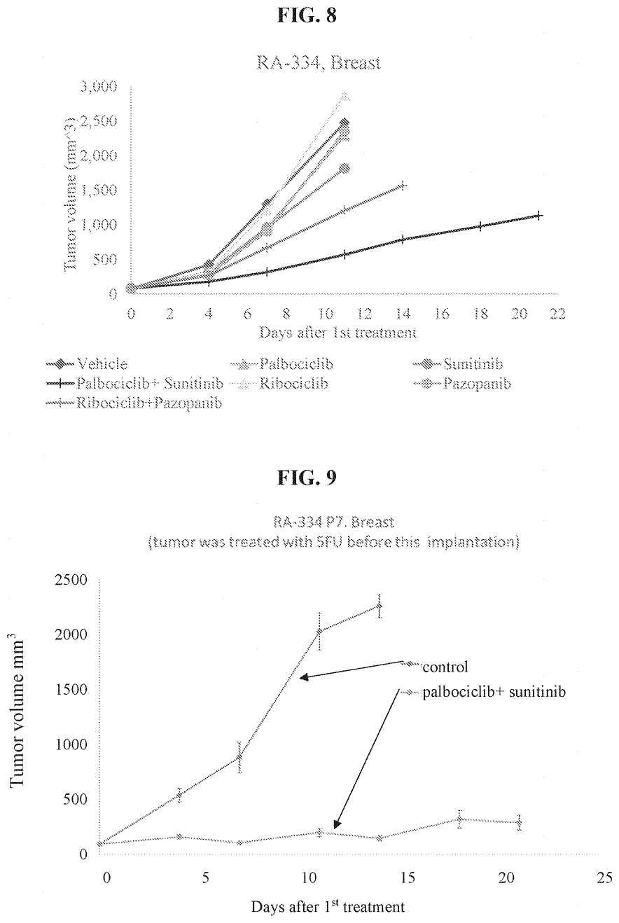

The results are presented in FIG. 9. It can be seen that tumor resistant to 5FU treatment was effectively treated with combination of palbociclib+sunitinib. This treatment completely prevented development and growth of 5FU resistant breast cancer.

It can be easily seen that the combo treatment is much more efficient that it could have been expected. In fact, the combo treatment completely prevented development of 5FU resistant breast cancer.

### Example 3C. Efficacy of the Palbociclib+Sunitinib Combo Treatment of Breast Cancer (RA-336)

Triple negative breast cancer (TNBC) tissue was grown in NRG mice, and further collected, dissociated and implanted in new NRG mice for drug efficacy study. When tumors reached a size of 60-210 mm³, mice were assigned to treatment groups based on the tumor size, tumor growth rate and body weight (BW), creating groups with similar average of these parameters (Table 16). Drugs dosing were initiated at the same day of randomization, denoted as Day 0. The experiment lasted 45 days. Tumor volume was measured.

TABLE 16
Study design

| Group | | # of treatments | Treatment | # of mice |
|---|---|---|---|---|
| # | Tested drugs | per week | method | per group |
| 1 | Control (vehicle) | 5 | PO | 6 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | Ribociclib 100 mg/kg | 5 | PO | 5 |
| 6 | Pazopanib 100 mg/kg | 5 | PO | 5 |
| 7 | Ribociclib 100 mg/kg + | 5 | PO | 5 |
| | Pazopanib 100 mg/kg | | | |
| 8 | Cisplatin 3mg/kg | 2 | IP | 4 |

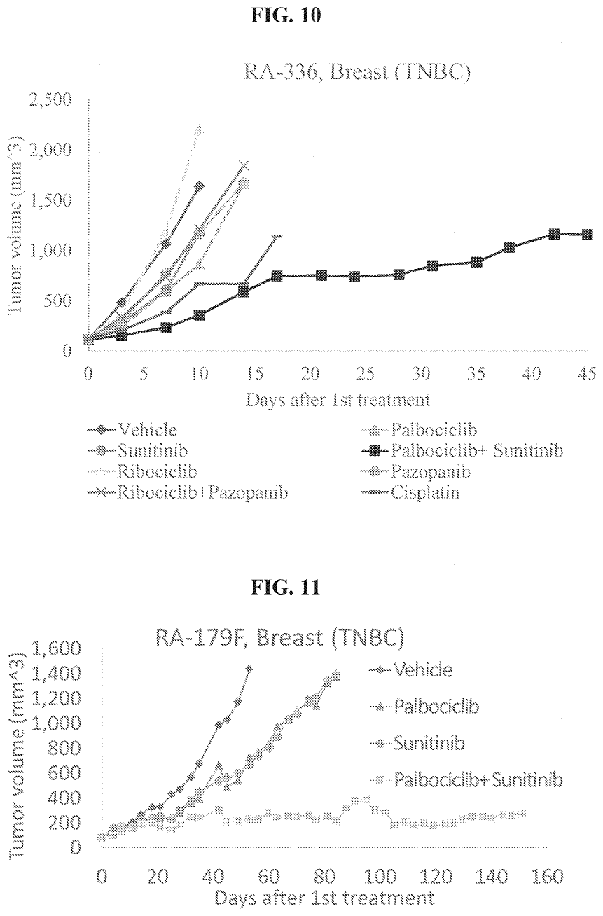

The results are presented in FIG. 10. It can be seen that each one of used drugs, i.e. palbociclib, sunitinib, ribociclib and pazopanib had weak to mild effect on tumor size. Combination of ribociclib and pazopanib did not show any additional effect over administration of pazopanib alone. On the contrary, the combination of palbociclib and sunitinib showed a synergic effect on TNBC breast cancer. The tumor developed much slower that one could expect from combination of the effects of each one of palbociclib and sunitinib. Moreover, it can be seen that the combo treatment with palbociclib and sunitinib was even more effective than the well-known treatment with cisplatin. The synergic effect is statistically significant as follows from P-values of Wilcoxon Test (Table 17) and from log rank value of survival test (Table 18).

TABLE 17
Wilcoxon Test P-values

| | | P-value, Wilcoxon |
|---|---|---|
| Treatment | Day | Test (Two-sided) |
| Palbociclib Vs combo | 10 | 0.0122 |
| Sunitinib Vs combo | 10 | 0.0367 |
| Mean Palbociclib | 10 | 0.0122 |
| Sunitinib Vs combo | | |
| Ribociclib Vs combo | 10 | 0.1437 |
| Pazopanib Vs combo | 10 | 0.6761 |
| Mean Ribociclib | 10 | 0.1437 |
| Pazopanib Vs combo | | |

TABLE 18
Log-Rank test

| | Log- |
|---|---|
| Treatment | Rank |
| Palbociclib Vs combo | 0.0158 |
| Sunitinib Vs combo | 0.0495 |

### Example 3D. Efficacy of the Palbociclib+Sunitinib Combo Treatment of Breast Cancer RA-179F

Triple negative breast cancer (TNBC) tissue from a different patient was grown in NRG mice, and further collected, dissociated and implanted in new NRG mice for drug efficacy study. When tumors reached a size of 60-160 mm³, mice were assigned to treatment groups based on the tumor size, tumor growth rate and body weight (BW), creating groups with similar average of these parameters (Table 19). Drugs dosing were initiated at the same day of randomization, denoted as Day 0. The experiment lasted 151 days. Tumor volume was measured.

TABLE 19
Study design

| Group | | # of treatments | Treatment | # of mice |
|---|---|---|---|---|
| # | Tested drugs | per week | method | per group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 6 |
| | Sunitinib 50 mg/kg | | | |

The results are presented in FIG. 11. It can be seen that each one of used drugs, i.e. palbociclib and sunitinib had a mild effect on tumor size. The combination of palbociclib and sunitinib showed a synergic effect on TNBC breast cancer. The tumor developed was almost completely inhibited for the whole length of the experiment. The synergic effect is statistically significant as follows from P-values of Wilcoxon Test (Table 20).

TABLE 20
Wilcoxon Test P-values

| | | P-value, Wilcoxon |
|---|---|---|
| Treatment | Day | Test (Two-sided) |
| Palbociclib Vs combo | 84 | 0.0081 |
| Sunitinib Vs combo | 84 | 0.0142 |
| Mean Palbociclib | 84 | 0.0081 |
| Sunitinib Vs combo | | |

### Example 3E. Efficacy of the Palbociclib+Sunitinib Combo Treatment of Breast Cancer (RA-405)

Breast cancer tissue from yet another patient was grown in NRG mice, and further collected, dissociated and implanted in new NRG mice for drug efficacy study. When tumors reached a size of 60-105 mm³, mice were assigned to treatment groups based on the tumor size, tumor growth rate and body weight (BW), creating groups with similar average of these parameters (Table 21). Drugs dosing were initiated at the same day of randomization, denoted as Day 0. The experiment lasted 77 days. Tumor volume was measured.

TABLE 21
Study Design

| Group | | # of treatments | Treatment | # of mice |
|---|---|---|---|---|
| # | Tested drugs | per week | method | per group |
| 1 | Control (vehicle) | 5 | PO | 6 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | 5-FU 30mg/kg | 2 | IP | 5 |

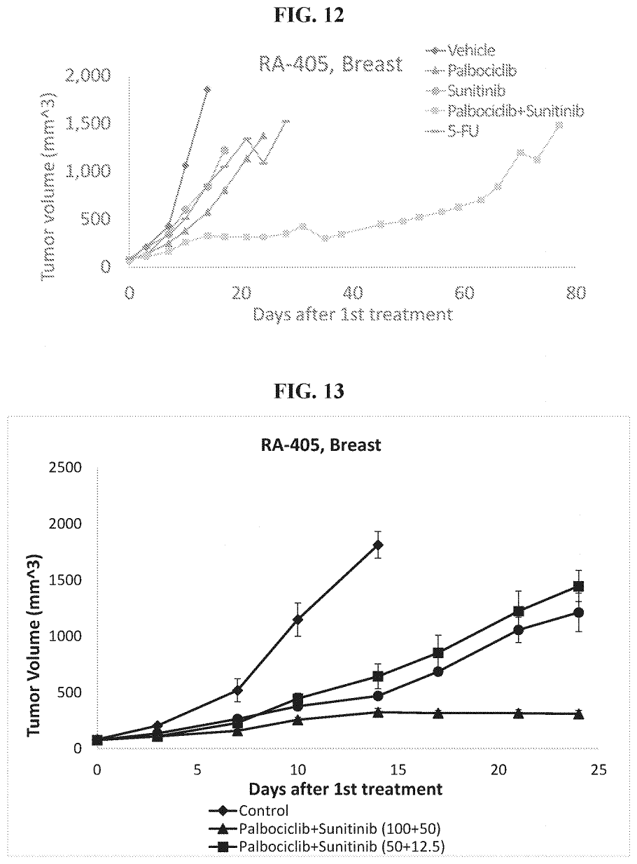

The results are presented in FIG. 12. It can be seen that each one of used drugs, i.e. palbociclib and sunitinib attenuate tumor growth. The combination of palbociclib and sunitinib showed a synergic effect on breast cancer. It inhibited tumor growth for more than 40 days. The synergic effect is statistically significant as follows from P-values of Wilcoxon Test (Table 22) and from log rank value of survival test (Table 23). The combo treatment was much more effective that 5-FU anti-tumor drug.

TABLE 22
Wilcoxon Test P-values

| | | P-value, Wilcoxon |
|---|---|---|
| Treatment | Day | Test (Two-sided) |
| Palbociclib Vs combo | 17 | 0.0122 |
| Sunitinib Vs combo | 17 | 0.0122 |
| Mean Palbociclib | 17 | 0.0122 |
| Sunitinib Vs combo | | |

TABLE 23
Log-rank values

| | Log- |
|---|---|
| Treatment | Rank |
| Palbociclib Vs combo | 0.0486 |
| Sunitinib Vs combo | 0.0495 |

Cancer sample from the same patient was used to elucidate the effect of dose on tumor size. The cancer tissue was treated as described above and implanted to mice according to Table 24.

TABLE 24
Study Design

| Group | | # of treatments | Treatment | # of mice |
|---|---|---|---|---|
| # | Tested drugs | per week | method | per group |
| 1 | Control (vehicle) | 5 | PO | 6 |
| 2 | Palbociclib 100 mg/kg + | 5 | PO | 5 |
| | Sunitinib 50 mg/kg | | | |
| 3 | Palbociclib 50 mg/kg + | 5 | PO | 5 |
| | Sunitinib 12.5 mg/kg | | | |
| 4 | Palbociclib 75 mg/kg + | 5 | PO | 5 |
| | Sunitinib 25 mg/kg | | | |

The results are presented in FIG. 13. On day 15 all doses show a significant inhibition of tumor growth compare to control. Tumor volume (mm³) of control=1800, max dose=325, mid dose=470, min. dose=644). On day 24, maximum doses of the combination results in continuous inhibition of tumor growth (Tv=310 mm³), while in the minimum and mid doses the tumors progress gradually. The combination of palbociclib+sunitinib has a dose response effect on tumor growth inhibition.

### Example 4A. Efficacy of the Palbociclib+Sunitinib Combo Treatment of Lung Cancer (RA-388)

Fresh tumor sample of poorly differentiated carcinoma was collected by biopsy from lung and grown in immunocompromised mice. Then the grown tumor cells were collected, dissociated and implanted subcutaneously to NRG mice necks as described in material and methods. When tumors reached a size of 60-160 mm³, mice were assigned to treatment groups based on the tumor size, tumor growth rate and body weight (BW), creating groups with similar average of these parameters (Table 25). Drugs dosing were initiated at the same day of randomization, denoted as Day 0. The experiment lasted 45 days. Tumor volume was measured.

TABLE 25
Study Design

| Group | | # of treatments | Treatment | # of mice |
|---|---|---|---|---|
| # | Tested drugs | per week | method | per group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | Palbociclib 100 mg/kg + | 5 | PO | 5 |
| | Sorafenib 20 mg/kg | | | |
| 6 | Sorafenib 20 mg/kg | 5 | PO | 5 |

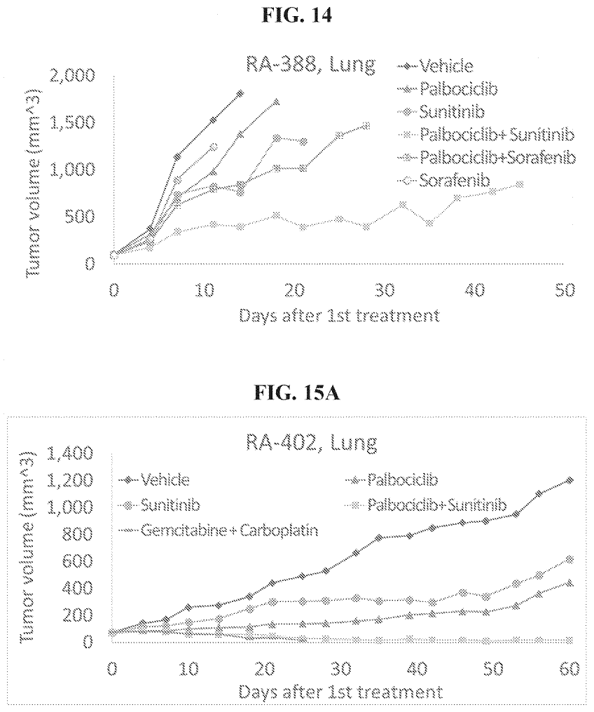

The results are presented in FIG. 14. It can be seen that each one of used drugs, i.e. palbociclib, sunitinib, and sorafenib had weak to mild effect on tumor size. Combination of palbociclib and sorafenib had a week synergic effect which was much more evident after day 14. Combination of palbociclib and sunitinib showed a strong synergic effect on lung cancer. The tumor developed much slower that one could expect from the combination of effects of each one of palbociclib and sunitinib. The synergic effect of palbociclib and sunitinib combination is statistically significant as follows from P-values of Wilcoxon Test (Table 26).

TABLE 26
Wilcoxon Test P-values

| | | P-value, Wilcoxon |
|---|---|---|
| Treatment | Day | Test (Two-sided) |
| Palbociclib Vs combo | 18 | 0.0200 |
| Sunitinib Vs combo | 18 | 0.0122 |
| Mean Palbociclib | 18 | 0.0122 |
| Sunitinib Vs combo | | |

### Example 4B. Efficacy of the Palbociclib+Sunitinib Combo Treatment of Lung Cancer (RA-402)

Fresh tumor sample of lung adenocarcinoma (KRAS mutation G12V) was collected by biopsy from lung and grown in immunocompromised mice. Then the grown tumor cells were collected, dissociated and implanted subcutaneously to NRG mice necks as described in material and methods. When tumors reached a size of 60-100 mm³, mice were assigned to treatment groups based on the tumor size, tumor growth rate and body weight (BW), creating groups with similar average of these parameters (Table 27). Drugs dosing were initiated at the same day of randomization, denoted as Day 0. The experiment lasted 45 days. Tumor volume was measured.

TABLE 27
Study Design

| Group | | # of treatments | Treatment | # of mice |
|---|---|---|---|---|
| # | Tested drugs | per week | method | per group |
| 1 | Control (vehicle) | 5 | PO | 6 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 6 |
| | Sunitinib 50 mg/kg | | | |
| 5 | Gemcitabine 25 mg/kg + | 2 | IP | 5 |
| | Carboplatin 50 mg/kg | 1 | | |

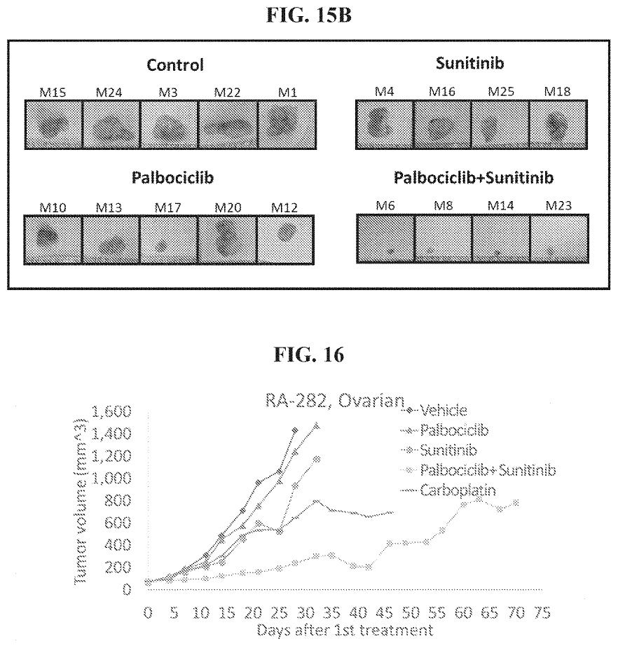

The results are presented in FIG. 15. It can be seen that both palbociclib and sunitinib effectively reduce tumor size (FIG. 15A). Nevertheless, with time left, the tumor processes and at day 50 both these treatments loose its effect. Combination of palbociclib and sorafenib had a much stronger effect and in fact, reduced the initial size of the tumor and completely prevented it proliferation. The co-administration of palbociclib and sunitinib showed a strong synergic effect on lung cancer. It was as effective as the SOC treatment with gencitabine+carboplatin. However, the combination of palbociclib and sunitinib was significantly safer to the mice compare to the SOC treatment. On day 25 the SOC group were excluded from the experiment due to toxicity with severe clinical signs (weakness and loss of weight), compare to the combination group in which all 6 mice were treated without any toxicity signs until day 60. The synergic effect of palbociclib and sunitinib combination is statistically significant as follows from P-values of Wilcoxon Test (Table 28). FIG. 15B show the tumors excised from the mice at day 60. It can be clearly seen that the tumors from the combination group are significantly smaller than all the other groups.

TABLE 28
Wilcoxon Test P-values

| | | P-value, Wilcoxon |
|---|---|---|
| Treatment | Day | Test (Two-sided) |
| Palbociclib Vs combo | 60 | 0.0195 |
| Sunitinib Vs combo | 60 | 0.0294 |
| Mean Palbociclib | 60 | 0.0195 |
| Sunitinib Vs combo | | |

### Example 5A. Efficacy of the Palbociclib+Sunitinib Combo Treatment of Ovarian Cancer (RA-282)

Fresh tumor sample of metastatic neuroendocrine ovarian cancer was grown in NRG mice, and further collected, dissociated and implanted in new NRG mice for drug efficacy study. When tumors reached a size of 60-120 mm³, mice were assigned to treatment groups based on the tumor size, tumor growth rate and body weight (BW), creating groups with similar average of these parameters (Table 29). Drugs dosing were initiated at the same day of randomization, denoted as Day 0. The experiment lasted 70 days. Tumor volume was measured.

TABLE 29
Study Design

| | | # of | | # of |
|---|---|---|---|---|
| Group | | treatments | Treatment | mice per |
| # | Tested drugs | per week | method | group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | Carboplatin 50 mg/kg | 2 | IP | 5 |

The results are presented in FIG. 16. It can be seen that the combination of palbociclib and sunitinib showed a strong synergic effect on ovarian cancer. Tumor growth was inhibited and stabilized until ˜day 42, by the combo treatment, after which they start developed resistance to therapy. The tumor developed much slower than one could expect from the combination of effects of each one of palbociclib and sunitinib. In fact, the combo treatment was even more efficient than the well know treatment with carboplatin. The synergic effect of palbociclib and sunitinib combination is statistically significant as follows from P-values of Wilcoxon Test (Table 30). In addition, in the carboplatin treatment group, the 3 out of 5 tumors formed ulcers between day 28 to 46, which led to termination of the study for this group at day 46, while in the combination group at day 46 only one mouse (out of 5) was excluded due to ulcer. Thus, the combo treatment did not only provide synergic treatment of the cancer, but was much safer than the SOC treatment.

TABLE 30
Wilcoxon Test P-values

| | | P-value, Wilcoxon |
|---|---|---|
| Treatment | Day | Test (Two-sided) |
| Palbociclib Vs combo | 32 | 0.0122 |
| Sunitinib Vs combo | 32 | 0.0369 |
| Mean Palbociclib | 32 | 0.0122 |
| Sunitinib Vs combo | | |

### Example 5B. Efficacy of the Palbociclib+Sunitinib Combo Treatment of Ovarian Cancer (RA-365)

Fresh tumor sample of metastatic high grade serous carcinoma with BRCA1 mutation (ovarian cancer) was grown in NRG mice, and further collected, dissociated and implanted in new NRG mice for drug efficacy study. When tumors reached a size of 60-150 mm³, mice were assigned to treatment groups based on the tumor size, tumor growth rate and body weight (BW), creating groups with similar average of these parameters (Table 31). Drugs dosing were initiated at the same day of randomization, denoted as Day 0. The experiment lasted 172 days. Tumor volume was measured. At day 172, 3 mice at the combo Palbociclib-Sunitinib group were still alive: 2 mice shows stable diseases, and one mouse with tumor that developed resistance and started to grow. In all groups there were 2-3 mice that removed from study due to ulcer or weakness, starting from day 38.

TABLE 31
Study Design

| | | # of | | # of |
|---|---|---|---|---|
| Group | | treatments | Treatment | mice per |
| # | Tested drugs | per week | method | group |
| 1 | Control (vehicle) | 5 | PO | 6 |
| 2 | Palbociclib 100 mg/kg | 5 | PO | 6 |
| 3 | Sunitinib 50 mg/kg | 5 | PO | 6 |
| 4 | Palbociclib 100 mg/kg + | 5 | PO | 6 |
| | Sunitinib 50 mg/kg | | | |
| 5 | Palbociclib 100 mg/kg + | 5 | PO | 5 |
| | Sorafenib 20 mg/kg | | | |
| 6 | Sorafenib 20 mg/kg | 5 | PO | 4 |

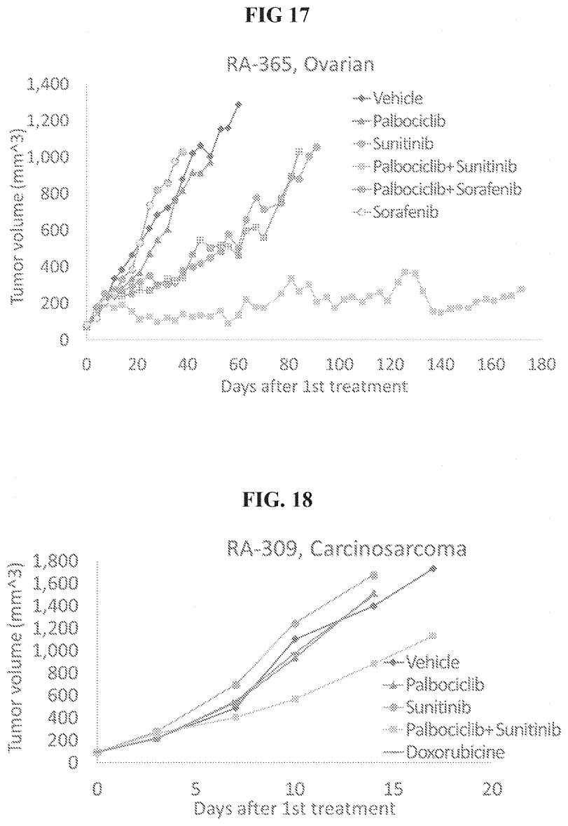

The results are presented in FIG. 17. It can be seen that treatment with palbociclib or sorafenib has no effect on tumor growth, sunitinib attenuates significantly tumor growth. It can be also seen that both combinations: palbociclib with sunitinib and palbociclib with sorafenib, both had a significant synergistic effect on ovarian tumor growth inhibition. It is evident that treatment with a combination of palbociclib and sunitinib is much more efficient than with a combination of palbociclib and sorafenib. The synergic effect of palbociclib and sunitinib combination is statistically significant as follows from P-values of Wilcoxon Test (Table 32). Due to technical problems the synergic effect cannot be expressed by statistical means, it is clearly seen on the figure and easily concluded from the trend.

TABLE 32
Wilcoxon Test P-values

| | | P-value, Wilcoxon |
|---|---|---|
| Treatment | Day | Test (Two-sided) |
| Palbociclib Vs combo | 49 | 0.0200 |
| Sunitinib Vs combo | 49 | 0.0137 |
| Mean Palbociclib | 49 | 0.0081 |
| Sunitinib Vs combo | | |
| Sunitinib Vs combo | 91 | 0.0369 |

### Example 6. Efficacy of the Palbociclib+Sunitinib Combo Treatment of Carcinosarcoma (RA-309)

Fresh tumor sample of carcinosarcoma was grown in NRG mice, and further collected, dissociated and implanted in new NRG mice for drug efficacy study. When tumors reached a size of 60-140 mm³, mice were assigned to treatment groups based on the tumor size, tumor growth rate and body weight (BW), creating groups with similar average of these parameters (Table 33). Drugs dosing were initiated at the same day of randomization, denoted as Day 0. The experiment lasted 17 days. Tumor volume was measured.

TABLE 33
Study Design

| | | # of | | # of |
|---|---|---|---|---|
| Group | | treatments | Treatment | mice per |
| # | Tested drugs | per week | method | group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | Doxorubicine 4 mg/kg | 1 | IP | 4 |

The results are presented in FIG. 18. It can be seen that none of the tested drugs when administered alone had any effect on tumor growth. However, in the combination group several mice responded very strongly to the treatment. It is thus evident that a combination of palbociclib with sunitinib inhibited tumor growth and provided profound synergistic inhibitory effect on carcinosarcoma. Although due to small group, it was not possible to show statistical evidence for the synergistic effect, it can be clearly seen from the graphs. It is also clear that the combo treatment is much more effective than the known anticancer drug doxorubicine.

### Example 7. Efficacy of the Palbociclib+Sunitinib Combo Treatment of Pancreatic Cancer (RA-347)

Fresh adenocarcinoma tumor (pancreatic cancer) was grown in NRG mice, and further collected, dissociated and implanted in new NRG mice for drug efficacy study. When tumors reached a size of 70-170, mice were assigned to treatment groups based on the tumor size, tumor growth rate and body weight (BW), creating groups with similar average of these parameters (Table 34). Drugs dosing were initiated at the same day of randomization, denoted as Day 0. The experiment lasted 66 days. Tumor volume was measured.

TABLE 34
Study Design

| | | # of | | # of |
|---|---|---|---|---|
| Group | | treatments | Treatment | mice per |
| # | Tested drugs | per week | method | group |
| 1 | Control (vehicle) | 5 | PO | 6 |
| 2 | Palbociclib 100 mg/kg | 5 | PO | 6 |
| 3 | Sunitinib 50 mg/kg | 5 | PO | 6 |
| 4 | Palbociclib 100 mg/kg + | 5 | PO | 6 |
| | Sunitinib 50 mg/kg | | | |
| 5 | Gemcitabine 25 mg/kg + | 2 | IP | 6 |
| | Cisplatin 3 mg/kg | | | |

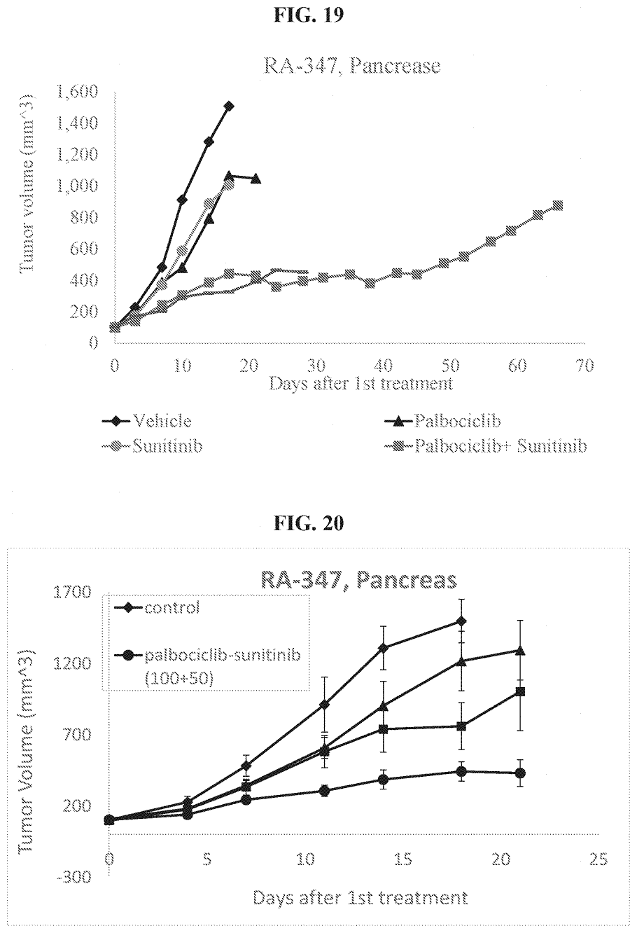

The results are presented in FIG. 19. It can be seen that palbociclib and sunitinib when administered alone had moderate effect on the tumor size. It is also evident that a combination of palbociclib with sunitinib significantly inhibited tumor growth for a prolonged period. It is also clear that the combo treatment has similar treatment efficacy power as a combination of two acknowledged anticancer drugs: gemcitabine and cisplatin (SOC treatment). However, the combination of palbociclib and sunitinib was significantly safer to the mice compare to the SOC treatment. On day 28 all mice in the SOC group were excluded from the experiment due to toxicity with severe clinical signs (weakness and loss of weight), compared to the combination group in which 4 out of 6 mice were treated without any toxicity signs until day 66. The synergic effect of palbociclib and sunitinib combination is statistically significant as follows from P-values of Wilcoxon Test (Table 35).

TABLE 35
Wilcoxon Test P-values

| | | P-value, Wilcoxon |
|---|---|---|
| Treatment | Day | Test (Two-sided) |
| Palbociclib Vs combo | 17 | 0.0131 |
| Sunitinib Vs combo | 17 | 0.0142 |
| Mean Palbociclib | 17 | 0.0082 |
| Sunitinib Vs combo | | |

The effect of Palbociclib+Sunitinib is dose dependent as has been further shown. Using samples obtained as discussed above and assigned to groups according to Table 36. The experiment lasted 21 days.

TABLE 36
Study Design

| | | # of | | # of |
|---|---|---|---|---|
| Group | | treatments | Treatment | mice per |
| # | Tested drugs | per week | method | group |
| 1 | Control (vehicle) | 5 | PO | 6 |
| 2 | Palbociclib 100 mg/kg + | 5 | PO | 6 |
| | Sunitinib 50 mg/kg | | | |
| 3 | Palbociclib 50 mg/kg + | 5 | PO | 6 |
| | Sunitinib 12.5 mg/kg | | | |
| 4 | Palbociclib 75 mg/kg + | 5 | PO | 6 |
| | Sunitinib 12.5 mg/kg | | | |

Results are presented in FIG. 20 and show clear dose dependence of the treatment. The highest tested dose almost completely inhibited tumor growth.

### Example 8. Efficacy of the Palbociclib+Sunitinib Combo Treatment of Cholangiocarcinoma (RA-372)

Fresh tumor sample of Cholangiocarcinoma was grown in NRG mice, and further collected, dissociated and implanted in new NRG mice for drug efficacy study. When tumors reached a size of 60-110 mm³, mice were assigned to treatment groups based on the tumor size, tumor growth rate and body weight (BW), creating groups with similar average of these parameters (Table 37). Drugs dosing were initiated at the same day of randomization, denoted as Day 0. The experiment lasted 87 days. Tumor volume was measured.

TABLE 37
Study Design

| | | # of | | # of |
|---|---|---|---|---|
| Group | | treatments | Treatment | mice per |
| # | Tested drugs | per week | method | group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | Gemcitabine 25 mg/kg + | 2 | IP | 4 |
| | Cisplatin 1.5 mg/kg | | | |

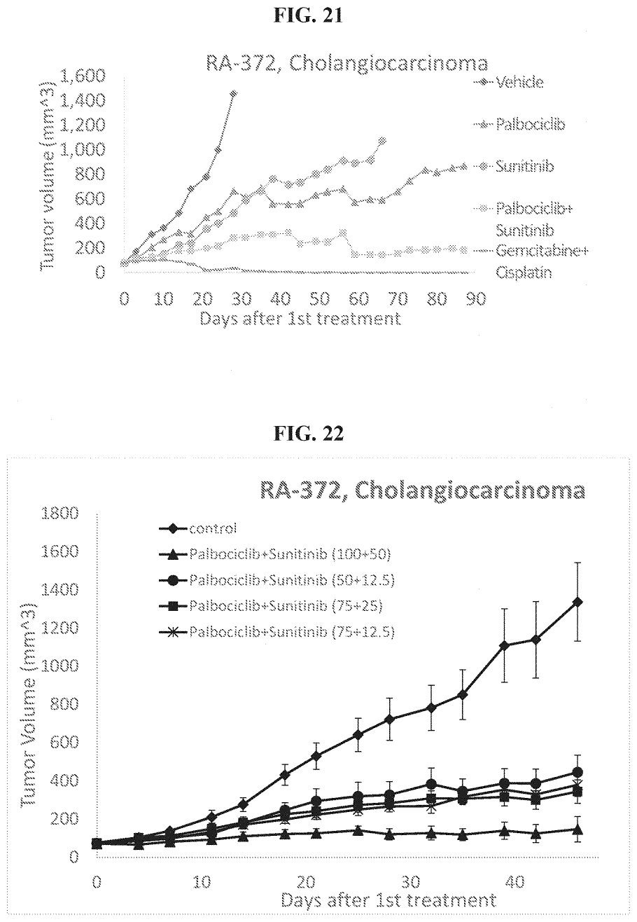

The results are presented in FIG. 21. It can be seen that treatment with each one of palbociclib and sunitinib provided similar moderate inhibitory effect on tumor growth. It can be also seen that the combination of palbociclib with sunitinib synergically inhibited tumor growth for the whole period of the experiment. In fact, the treatment with a combination of palbociclib and sunitinib provided similar results to those obtained from the combination of two known anticancer drugs: gemcitabine and cisplatin. The synergic effect of palbociclib and sunitinib combination is statistically significant as follows from P-values of Wilcoxon Test (Table 38).

TABLE 38
Wilcoxon Test P-values

| | | P-value, Wilcoxon |
|---|---|---|
| Treatment | Day | Test (Two-sided) |
| Palbociclib Vs combo | 66 | 0.0200 |
| Sunitinib Vs combo | 66 | 0.0373 |
| Mean Palbociclib | 66 | 0.0200 |
| Sunitinib Vs combo | | |

Dose response was tested for 46 days. The mice were allocated according to Table 39.

TABLE 39
Study Design

| | | # of | | # of |
|---|---|---|---|---|
| Group | | treatments | Treatment | mice per |
| # | Tested drugs | per week | method | group |
| 1 | Control (vehicle) | 5 | PO | 6 |
| 2 | Palbociclib 100 mg/kg + | 5 | PO | 7 |
| | Sunitinib 50 mg/kg | | | |
| 3 | Palbociclib 50 mg/kg + | 5 | PO | 7 |
| | Sunitinib 12.5 mg/kg | | | |
| 4 | Palbociclib 75 mg/kg + | 5 | PO | 6 |
| | Sunitinib 25 mg/kg | | | |
| 5 | Palbociclib 75 mg/kg + | 5 | PO | 6 |
| | Sunitinib 12.5 mg/kg | | | |

Results shown in FIG. 22 clearly show dose dependence of the treatment. All tested combinations were highly effective, while the highest dose regiment provided the most profound effect in inhibiting tumor growth.

### Example 9. Efficacy of the Palbociclib+Sunitinib Combo Treatment of Hepatocellular (RA-374)

Fresh tumor sample hepatitis C (HCV) positive hepatocellular carcinoma was grown in NRG mice, and further collected, dissociated and implanted in new NRG mice for drug efficacy study. When tumors reached a size of 60-260 mm³, mice were assigned to treatment groups based on the tumor size, tumor growth rate and body weight (BW), creating groups with similar average of these parameters (Table 40). Drugs dosing were initiated at the same day of randomization, denoted as Day 0. The experiment lasted 14 days. Tumor volume was measured.

TABLE 40
Study Design

| | | # of | | # of |
|---|---|---|---|---|
| Group | | treatments | Treatment | mice per |
| # | Tested drugs | per week | method | group |
| 1 | Control (vehicle) | 5 | PO | 7 |
| 2 | Palbociclib 100 mg/kg | 5 | PO | 7 |
| 3 | Sunitinib 50 mg/kg | 5 | PO | 8 |
| 4 | Palbociclib 100 mg/kg + | 5 | PO | 7 |
| | Sunitinib 50 mg/kg | | | |
| 5 | Sorafenib 20mg/kg | 5 | PO | 6 |

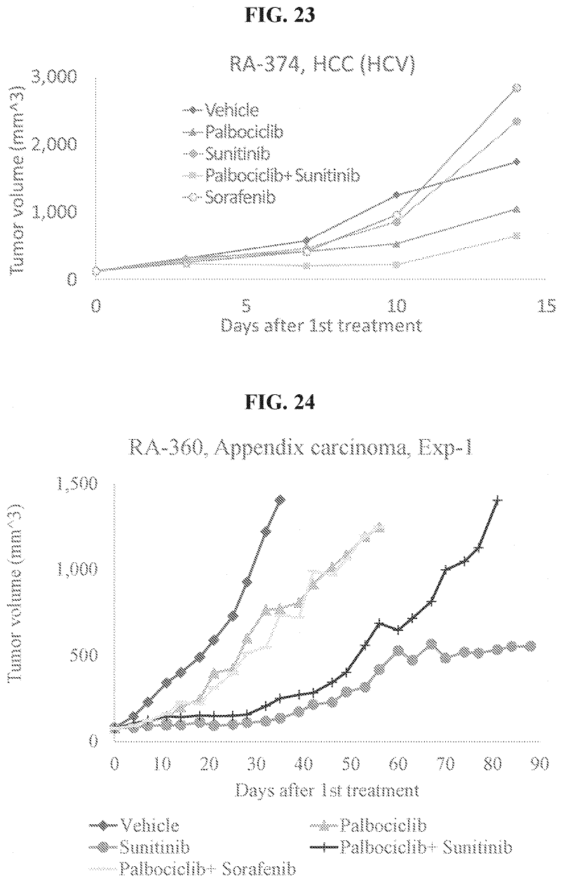

The results are presented in FIG. 23. It can be seen that treatment with a combination of palbociclib and sunitinib was only slightly better than that obtained with palbociclib alone. No synergic effect was seen for the combination as also evident from the high P-values of Wilcoxon Test (Table 41).

TABLE 41
Wilcoxon Test P-values

| | | P-value, Wilcoxon |
|---|---|---|
| Treatment | Day | Test (Two-sided) |
| Palbociclib Vs combo | 14 | 0.6171 |
| Sunitinib Vs combo | 14 | 0.0306 |
| Mean Palbociclib | 14 | 0.0169 |
| Sunitinib Vs combo | | |

### Example 10. Efficacy of the Palbociclib+Sunitinib Combo Treatment of Appendix Carcinoma (RA-360)

Fresh tumor sample of appendix carcinoma was grown in NRG mice, and further collected, dissociated and implanted in new NRG mice for drug efficacy study. When tumors reached a size of 60-140 mm³, mice were assigned to treatment groups based on the tumor size, tumor growth rate and body weight (BW), creating groups with similar average of these parameters (Table 42). Drugs dosing were initiated at the same day of randomization, denoted as Day 0. The experiment lasted 35 days. Tumor volume was measured.

TABLE 42
Study Design

| | | # of | | # of |
|---|---|---|---|---|
| Group | | treatments | Treatment | mice per |
| # | Tested drugs | per week | method | group |
| 1 | Control (vehicle) | 5 | PO | 10 |
| 2 | Palbociclib 100 mg/kg | 5 | PO | 6 |
| 3 | Sunitinib 50 mg/kg | 5 | PO | 5 |
| 4 | Palbociclib 100 mg/kg + | 5 | PO | 6 |
| | Sunitinib 50 mg/kg | | | |
| 5 | Palbociclib 100 mg/kg + | 5 | PO | 5 |
| | Sorafenib 20 mg/kg | | | |
| 6 | Ribociclib 100 mg/kg | 5 | PO | 6 |
| 7 | Pazopanib | 5 | PO | 6 |
| 8 | Ribociclib 100 mg/kg + | 5 | PO | 6 |
| | Pazopanib 100 mg/kg | | | |

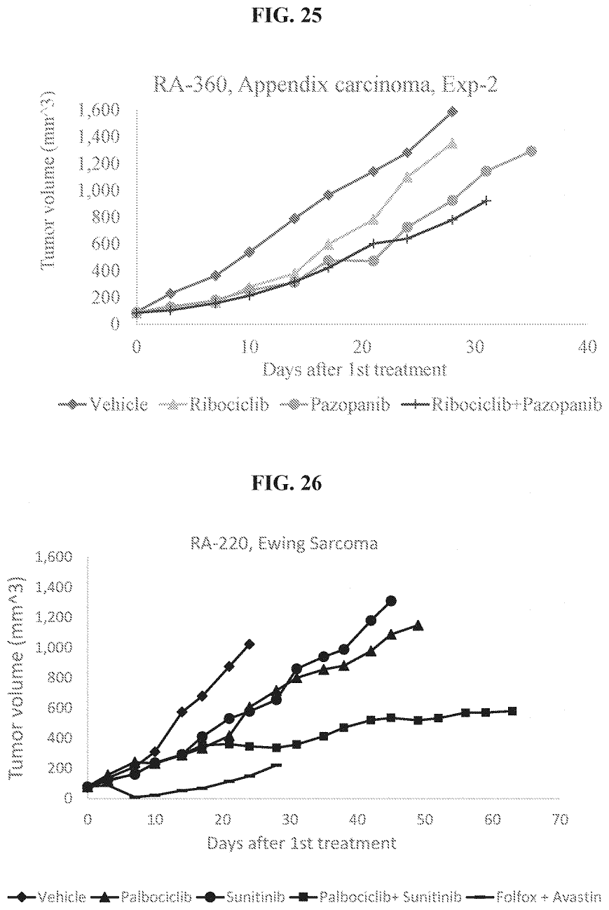

The results are presented in FIGS. 24 and 25. It can be seen that none of the combinations, i.e. palbociclib and sunitinib, palbociclib and sorafenib, and ribociclib and pazopanib had a synergic effect. In fact, sunitinib alone worked better than its combination with palbociclib. It seems that sorafenib in combination with palbociclib had no effect as the combo behaves exactly as palbociclib alone. Combination of ribociclib and pazopanib behaved as pazopanib alone. It is clear that in this type of cancer, the combo treatment had no added value over separate administrations.

### Example 11. Efficacy of the Palbociclib+Sunitinib Combo Treatment of Ewing Sarcoma (RA-220)

Fresh tumor sample of Ewing Sarcoma was grown in NRG mice, and further collected, dissociated and implanted in new NRG mice for drug efficacy study. When tumors reached a size of 60-100 mm³, mice were assigned to treatment groups based on the tumor size, tumor growth rate and body weight (BW), creating groups with similar average of these parameters (Table 43). Drugs dosing were initiated at the same day of randomization, denoted as Day 0. The experiment lasted 35 days. Tumor volume was measured.

TABLE 43
Study Design

| | | # of | | # of |
|---|---|---|---|---|
| Group | | treatments | Treatment | mice per |
| # | Tested drugs | per week | method | group |
| 1 | control (vehicle) | 5 | PO | 5 |
| 2 | palbociclib 100 mg/kg | 5 | PO | 5 |
| 3 | sunitinib 50 mg/kg | 5 | PO | 5 |
| 4 | palbociclib 100 mg/kg + | 5 | PO | 5 |
| | sunitinib 50 mg/kg | | | |
| 5 | ifosfamid 60 mg/kg+ | 2 | IP | 6 |
| | doxorubicine 4 mg/kg | 1 | | |

The results are presented in FIG. 26. It can be seen that palbociclib and sunitinib when administered alone had moderate effect on the tumor size. It is also evident that a combination of palbociclib with sunitinib significantly inhibited tumor growth for a prolonged period. The combo treatment has lower efficacy the treatment with ifosfamid with doxorubicine (SOC). Nevertheless, with time going it seen that the combo treatment was equal to it in efficiency. Not less important factor is that the combo treatment was much less toxic than the SOC. Mice of SOC treatment were very weak and lose weight, and started to be excluded from the study from day 7 to day 42. On the contrary, all mice of the combo treatment reached the end of the experiment.

Although the present invention has been described herein above by way of preferred embodiments thereof, it can be modified, without departing from the spirit and nature of the subject invention as defined in the appended claims.

1. A method for treating a solid caner, comprising administering to a subject in need thereof a combination of an inhibitor of cyclin-dependent kinase 4/6 (CDK 4/6) and a multi-targeted receptor tyrosine kinase inhibitor (mtRTKI), wherein the CDK 4/6 is palbociclib and mtRTKI is at least one of sunitinib or sorafenib, provided that the cancer is not hepatocellular carcinoma.

2. The method according to claim 1, wherein the combination comprises palbociclib and sunitinib.

3. The method according to claim 1, wherein the combination comprises palbociclib and sorafenib.

4. The method according to claim 1, wherein the solid cancer is at least one of: carcinoma, neuroendocrine tumor, carcinosarcoma, sarcoma, Ewing sarcoma, lymphoma, or melanoma.

5. The method according to claim 4, wherein the cancer is carcinoma.

6. The method according to claim 5, wherein the carcinoma is adenocarcinoma.

7. The method according claim 4, wherein the cancer is at least one of: lung cancer, stomach cancer, breast cancer, ovarian cancer, colon cancer, neuroendocrine cancer, pancreas cancer, cholangiocarcinoma, bone cancer, liposarcoma or adrenal cancer.

8. (canceled)

9. The method according to claim 1, characterized by at least one of:
(i) palbociclib is administered in a dose of from 20 to 250 mg/day and sunitinib is administered in a dose of from 10 to 125 mg/day;
(ii) palbociclib is administered in a dose of 20 to 250 mg/day and sorafenib is administered in a dose of 200 to 800 mg/day;
(iii) the CDK 4/6 inhibitor and the mtRTKI are administered in a sequential manner or in a substantially simultaneous manner; or
(iv) the combination administered provides a synergistic anti-cancer effect.

10-20. (canceled)

21. A pharmaceutical composition comprising an inhibitor of cyclin-dependent kinase 4/6 (CDK 4/6) and a multi-targeted receptor tyrosine kinase inhibitor (mtRTKI), wherein the CDK 4/6 inhibitor is selected from the group consisting of palbociclib, abemaciclib and ribociclib and the mtRTKI is selected from the group consisting of sunitinib, sorafenib and pazopanib.

22. The pharmaceutical composition of claim 21, comprising palbociclib and sunitinib.

23. The pharmaceutical composition of claim 22, comprising from 20 to 150 mg palbociclib and from 5 to 75 mg of sunitinib.

24. The pharmaceutical composition of claim 21, comprising palbociclib and sorafenib.

25. The pharmaceutical composition of claim 24, comprising from 20 to 150 mg wherein palbociclib and from 50 to 800 mg of sorafenib

26. The pharmaceutical composition of claim 21, comprising ribociclib and pazopanib.

27. The pharmaceutical composition of claim 26, comprising from 50 to 300 mg ribociclib and from 100 to 500 mg of pazopanib.

28. The pharmaceutical composition of claim 21, comprising abemaciclib and sorafenib.

29. The pharmaceutical composition of claim 28, comprising from 20 to 300 mg abemaciclib and from 50 to 800 mg of sorafenib.

30-42. (canceled)

43. A method for treating solid cancer, comprising administering to a subject in need thereof an inhibitor of cyclin-dependent kinase 4/6 (CDK 4/6) and a multi-targeted receptor tyrosine kinase inhibitor (mtRTKI), wherein the CDK 4/6 inhibitor is selected from the group consisting of palbociclib, abemaciclib and ribociclib, and the mtRTKI is selected from the group consisting of sunitinib, sorafenib and pazopanib, provided that: (i) palbociclib is not administered in combination with sorafenib, or abemaciclib is not administered in combination with sunitinib, or (ii) the cancer is not hepatocellular carcinoma or renal cell carcinoma.

44. The method of claim 43, characterized by at least one of:
(i) administering a combination of palbociclib and sunitinib;
(ii) administering a combination of palbociclib and sorafenib for treatment of solid cancer that is not hepatocellular carcinoma or renal cell carcinoma;
(iii) administering a combination of ribociclib and pazopanib;
(iv) administering a combination of abemaciclib and sorafenib; or
(v) the method provides a synergistic anti-cancer effect.

45. The method of claim 42, wherein the cancer is at least one of lung cancer, stomach cancer, breast cancer, ovarian cancer, colon cancer, neuroendocrine cancer, pancreas cancer, cholangiocarcinoma, bone cancer, liposarcoma, or adrenal cancer.
